(19) United States
(12) Patent Application Publication
(10) Pub. No.: US 2005/0084931 A1
(43) Pub. Date: Apr. 21, 2005

(54) Human prostaglandin DP receptor variants and methods of using same

(76) Inventors: Yanbin Liang, Irvine, CA (US); David F. Woodward, Lake Forest, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 10/689,861

(22) Filed: Oct. 20, 2003

(51) Int. Cl. C07K 14/705; C07H 21/04

(52) U.S. Cl. 435/69.1; 435/320.1; 435/325; 530/350; 536/23.5

(57) ABSTRACT

The present invention relates to a nucleic acid molecule encoding a novel, alternatively spliced DP receptor variant. A variant DP polypeptide and screening methods based on such a polypeptide also are provided.

### BACKGROUND OF THE INVENTION

1. Field of the Invention

This invention relates generally to molecular medicine and, more specifically, to alternatively spliced prostaglandin DP receptors.

2. Background Information

Prostaglandins (PG) and thromboxane, collectively named prostanoids, are oxygenated fatty acids that bind to seven transmembrane domain G-protein coupled receptors (GPCRs). The classification of prostanoid receptors into DP, EP, FP, IP, and TP is based on the binding and functional potency of the five naturally occurring prostanoids, PGD₂, PGE₂, PGF_{2α}, I₂, and TXA₂, respectively. Prostanoid receptors have been cloned and expressed in cultured cells, where ligand binding and signal transduction properties have been studied. It is recognized that prostanoids can bind to more than one prostanoid receptor type; however, each prostanoid binds to its respective receptor with an affinity at least one order of magnitude higher than its affinity for the other four prostanoid receptors.

Prostanoids produce numerous physiologic and pathophysiologic effects and regulate cellular processes in nearly every tissue. The wide spectrum of prostanoid action includes effects on immune, endocrine, cardiovascular, renal and reproductive systems as well as the contraction and relaxation of smooth muscle. Accordingly, prostanoids and prostanoid analogues have been used as drugs to treat a variety of clinical conditions, including, but not limited to, various types of pain.

The DP receptor, the least abundant of the prostanoid receptors, is expressed at low levels in most tissues and is highly expressed in leptomeninges, retina, and mucus-secreting cells of the gastrointestinal tract. DP receptors also may be localized in platelets; neutrophils; non-chromaffin cells from adrenal medulla; smooth muscle cells from several tissues; and nerve cells including cells of the central nervous system. Roles for DP receptors have been suggested in pain, sleep regulation and treatment of glaucoma. Recently, a role for the DP receptor in allergic disorders such as allergic asthma has been confirmed by genetic studies using a mouse targeted gene disruption model.

Although a few ligands such as BW245C are selective for the DP receptor, many compounds are non-selective, acting at multiple PG receptor subtypes. Thus, while a limited number of compounds have been synthesized that have reduced agonist activity at other prostanoid receptors, most currently available ligands suffer from a lack of receptor specificity, which can result in undesirable side effects.

One goal of clinical pharmacology is the development of more selective drugs with greater efficacy and fewer side effects than those currently available. Newly identified DP receptors such as alternatively spliced DP receptors can be more closely associated with a particular condition than the known DP receptor and can, therefore, be valuable targets for drug discovery efforts. This can result in the development of drugs having greater efficacy or fewer side effects than drugs developed against wild-type DP receptor.

Thus, there exists a need for the discovery of new DP receptors which can be used, for example, to design more specific drugs with fewer side effects. The present invention satisfies this need and provides related advantages as well.

### SUMMARY OF THE INVENTION

The present invention provides an isolated polypeptide that contains an amino acid sequence having at least 50% amino acid identity with SEQ ID NO: 4, and the amino acid sequence of SEQ ID NO: 5 or 6; or a conservative variant thereof. An isolated polypeptide of the invention can include, for example, an amino acid sequence having at least 80% amino acid identity with SEQ ID NO: 4, or an amino acid sequence having at least 90% amino acid identity with SEQ ID NO: 4. In one embodiment, the invention provides an isolated polypeptide that contains the amino acid sequence of SEQ ID NO: 2, or a conservative variant thereof. In other embodiments, the invention provides an isolated polypeptide containing the amino acid sequence of SEQ ID NO: 2, or consisting of SEQ ID NO: 2. Cells which contain any of the above exogenously expressed polypeptides also are provided by the invention.

The invention additionally provides a method for identifying a compound that modulates a DP receptor variant by contacting an isolated DP receptor variant or a DP receptor variant over-expressed in a genetically engineered cell with a compound, and determining the level of an indicator which correlates with modulation of the DP receptor variant, where an alteration in the level of the indicator as compared to a control level indicates that the compound is a compound that modulates the DP receptor variant and where the DP receptor variant is a polypeptide containing an amino acid sequence having at least 50% amino acid identity with SEQ ID NO: 4, and the amino acid sequence of SEQ ID NO: 5 or 6 or a conservative variant thereof. The alteration can be, for example, an increase in the level of the indicator or a decrease in the level of the indicator. In one embodiment, a method of the invention is practiced with a DP receptor variant that contains an amino acid sequence having at least 80% amino acid identity with SEQ ID NO: 4. In another embodiment, a method of the invention is practiced with a DP receptor variant that contains the amino acid sequence of SEQ ID NO: 2, or a conservative variant thereof. In a further embodiment, a method of the invention is practiced with a DP receptor variant which is an isolated polypeptide. In yet a further embodiment, a method of the invention is practiced with a DP receptor variant over-expressed in a genetically engineered cell. Indicators useful in the methods of the invention include, without limitation, calcium, and compounds to be screened according to a method of the invention include, but are not limited to, polypeptides and small molecules.

Further provided herein is a method for identifying a compound that specifically binds to a DP receptor variant by contacting an isolated DP receptor variant or a DP receptor variant over-expressed in a genetically engineered cell with a compound, and determining specific binding of the compound to the DP receptor variant, where the DP receptor variant is a polypeptide containing an amino acid sequence having at least 50% amino acid identity with SEQ ID NO: 4, and the amino acid sequence of SEQ ID NO: 5 or 6 or a conservative variant of SEQ ID NO: 5 or 6.

In one embodiment, a method of the invention is practiced with a DP receptor variant that contains an amino acid sequence having at least 80% amino acid identity with SEQ ID NO: 4. In another embodiment, a method of the invention is practiced with a DP receptor variant that contains the amino acid sequence of SEQ ID NO: 2, or a conservative variant thereof. In a further embodiment, a method of the invention is practiced with a DP receptor variant which is an isolated polypeptide. In yet a further embodiment, a method of the invention is practiced with a DP receptor variant over-expressed in a genetically engineered cell. In a screening method of the invention, the alteration can be, for example, an increase in the level of the indicator or a decrease in the level of the indicator. Indicators useful in the methods of the invention include, without limitation, calcium. Furthermore, compounds to be screened according to a method of the invention include, but are not limited to, polypeptides and small molecules.

The invention further provides a method for identifying a compound that differentially modulates a DP receptor variant by contacting an isolated DP receptor variant or a DP receptor variant over-expressed in a genetically engineered cell with a compound; determining the level of an indicator which correlates with modulation of the DP receptor variant; contacting a second receptor with the compound; determining the level of a corresponding indicator which correlates with modulation of the second receptor; and comparing the level of the indicator which correlates with modulation of the DP receptor variant with the level of the corresponding indicator which correlates with modulation of the second receptor, where a different level of the indicator which correlates with modulation of the DP receptor variant as compared to the level of the corresponding indicator indicates that the compound is a compound that differentially modulates the DP receptor variant and where the DP receptor variant is a polypeptide containing an amino acid sequence having at least 50% amino acid identity with SEQ ID NO: 4, and the amino acid sequence of SEQ ID NO: 5 or 6 or a conservative variant of SEQ ID NO: 5 or 6.

In a screening method of the invention, the second receptor can have, for example, the amino acid sequence of SEQ ID NO: 4 or a functional fragment thereof. DP receptor variants useful in the invention include polypeptides containing an amino acid sequence having at least 80% amino acid identity with SEQ ID NO: 4, and polypeptides containing the amino acid sequence of SEQ ID NO: 2, or a conservative variant thereof. DP receptor variants useful in the invention further include isolated polypeptides, and polypeptides over-expressed in a genetically engineered cell. In a screening method of the invention, the alteration can be, for example, an increase in the level of the indicator or a decrease in the level of the indicator. Indicators useful in the methods of the invention include, without limitation, calcium, and compounds to be screened according to a method of the invention include, but are not limited to, polypeptides and small molecules.

The present invention additionally provides a method for identifying a compound that differentially binds to a DP receptor variant by contacting an isolated DP receptor variant or a DP receptor variant over-expressed in a genetically engineered cell with a compound; determining specific binding of the compound to the DP receptor variant; contacting a second receptor with the compound; determining specific binding of the compound to the second receptor; and comparing the level of specific binding to the DP receptor variant with the level of specific binding to the second receptor, where a different level of specific binding to the DP receptor variant as compared to the level of specific binding to the second receptor indicates that the compound is a compound that differentially binds to a DP receptor variant and where the DP receptor variant is a polypeptide containing an amino acid sequence having at least 50% amino acid identity with SEQ ID NO: 4, and the amino acid sequence of SEQ ID NO: 5 or 6 or a conservative variant of SEQ ID NO: 5 or 6. A variety of second receptors are useful in the methods of the invention including, without limitation, those having the amino acid sequence of SEQ ID NO: 4 or a functional fragment thereof. DP receptor variants useful in the invention include, but are not limited to, polypeptides containing an amino acid sequence having at least 80% amino acid identity with SEQ ID NO: 4, and polypeptides containing the amino acid sequence of SEQ ID NO: 2, or a conservative variant thereof. Such DP receptor variants include isolated polypeptides as well as polypeptides over-expressed in a genetically engineered cell. Indicators useful in the methods of the invention include, without limitation, calcium, as well as indicators of specific binding to cells or isolated receptors. Compounds to be screened according to a method of the invention include, but are not limited to, polypeptides and small molecules.

### BRIEF DESCRIPTION OF THE DRAWINGS

FIG. 1 shows the nucleotide sequence of DP receptor variant VAR-1 (SEQ ID NO: 1). The underlined sequence indicates novel nucleotide sequence compared to the nucleotide sequence of the known wild-type human DP receptor (SEQ ID NO: 3). The start and stop codons for DP receptor variant VAR-1 are indicated in bold.

FIG. 2 shows a comparison of the amino acid sequences of the known wild-type human DP receptor (SEQ ID NO: 4), abbreviated as WT DP, with human DP receptor variant DP VAR-1 (SEQ ID NO: 2). An arrow in the carboxy terminal area of the polypeptides indicates the location where the sequence of the wild-type human DP receptor and the DP receptor variant DP VAR-1 begin to differ.

FIG. 3 shows distribution of mRNA from DP receptor variant DP VAR-1 in various tissues using RT-PCR. The location of the DP VAR-1 and wild type DP receptor (WT DP) mRNAs are indicated by arrows.

FIG. 4 shows a comparison of the genomic structure of the DP receptor gene and the human DP variant VAR-1. DP receptor variant DP VAR-1 retains “exon A,” which is an internal portion of the intron spliced out of the wild-type DP receptor.

### DETAILED DESCRIPTION OF THE INVENTION

The present invention is directed to the exciting discovery of novel DP receptor variants. DP receptor variants can be used to determine and refine the specificity of binding of compounds that bind to the known wild-type DP receptor. DP receptor variants also can be used to identify compounds that differentially modulate or bind to a first DP receptor variant in relation to wild-type DP receptor or a second DP receptor variant.

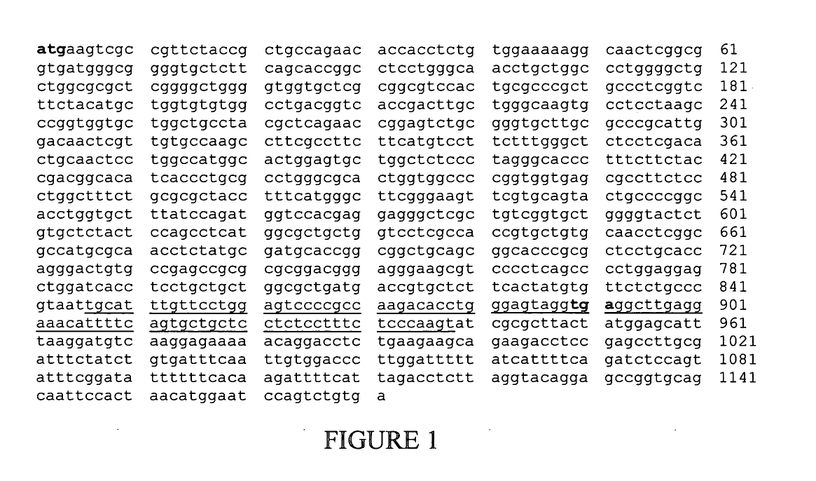

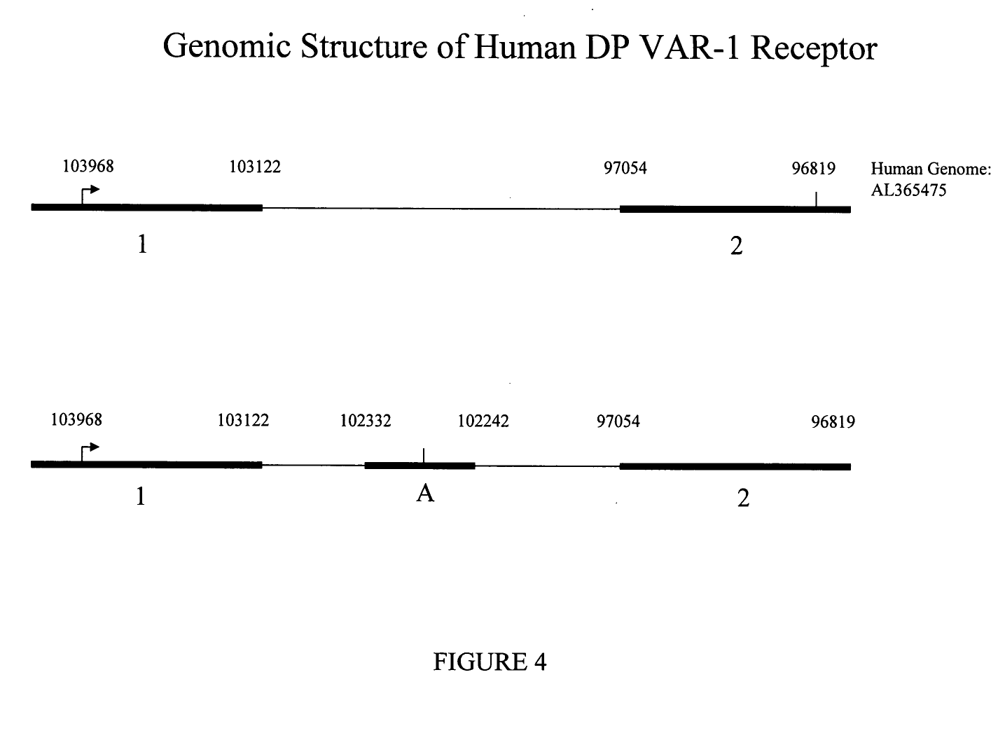

As disclosed herein in Example I, a novel DP receptor variant, DP VAR-1, was identified using the reverse transcription polymerase chain reaction (RT-PCR) and the following DP receptor primers: TGATGACCGTGCTCTTCACT (SEQ ID NO: 11) and GATAGAAATCGCAAGGCTCG (SEQ ID NO: 12). This novel alternatively spliced DP receptor variant DP VAR-1 contains an additional exon, designated “exon A,” as compared to the wild type DP receptor transcript (see FIGS. 1 and 4).

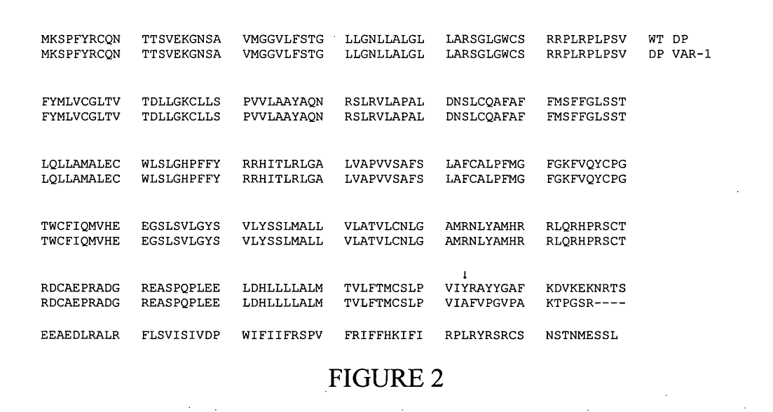

As further disclosed herein, sequence analysis of the nucleic acid molecule encoding the alternatively spliced DP receptor variant DP VAR-1 revealed novel carboxy-terminal amino acid sequence. The additional exon in DP receptor variant DP VAR-1 causes a frameshift and premature stop codon which results in different amino acid sequence from the wild-type human DP receptor. As shown in FIG. 2, the novel, shortened carboxy-terminal sequence of the DP receptor variant VAR-1 is AFVPGVPAKTPGSR (SEQ ID NO: 6). Furthermore, comparison of the known wild-type human DP receptor amino acid sequence (SEQ ID NO: 4) to the alternatively spliced human DP receptor variant DP VAR-1 sequence (SEQ ID NO: 2) revealed the amino acid sequence at the junction between conserved exon 1 and exon A within DP receptor variant DP VAR-1 to be LPVIAFVPGV (SEQ ID NO: 5), where the first four amino acids correspond to amino acid sequence present in conserved exon 1 and the remaining six amino acids are residues derived from exon A.

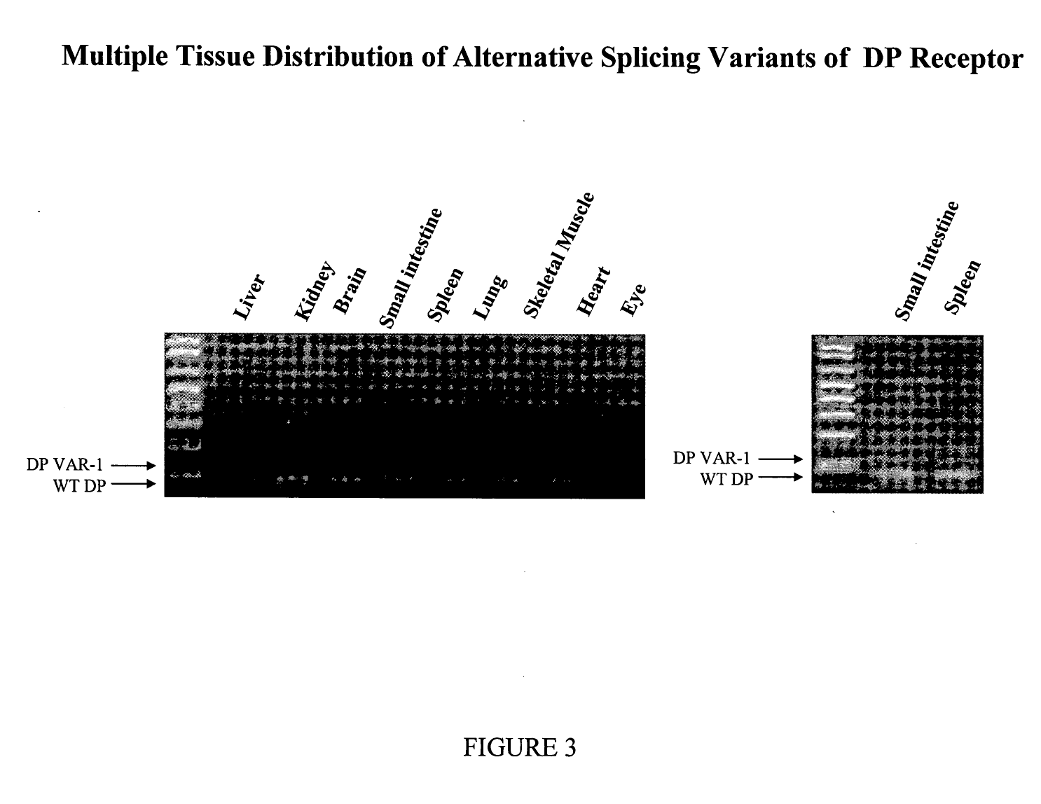

Also disclosed herein in Example II is the tissue distribution pattern of DP receptor variant DP VAR-1 (see FIG. 3). RT-PCR analysis was performed using the DP VAR-1-specific primers SEQ ID NOS: 11 and 12. As shown in FIG. 3, DP receptor variant DP VAR-1 mRNA, slightly smaller than wild type DP receptor transcript, was tissue specifically expressed in small intestine and spleen.

Based on these discoveries, the present invention provides novel alternatively spliced DP receptor variants and screening methods that rely on these variants. In particular, the present invention provides an isolated polypeptide that contains an amino acid sequence having at least 50% amino acid identity with SEQ ID NO: 4, and the amino acid sequence of SEQ ID NO: 5 or 6; or a conservative variant thereof. An isolated polypeptide of the invention can include, for example, an amino acid sequence having at least 80% amino acid identity with SEQ ID NO: 4, or an amino acid sequence having at least 90% amino acid identity with SEQ ID NO: 4. In one embodiment, the invention provides an isolated polypeptide that contains the amino acid sequence of SEQ ID NO: 2, or a conservative variant thereof. In other embodiments, the invention provides an isolated polypeptide containing the amino acid sequence of SEQ ID NO: 2, or consisting of SEQ ID NO: 2. Cells which contain any of the above exogenously expressed polypeptides also are provided by the invention.

The invention additionally provides a method for identifying a compound that modulates a DP receptor variant by contacting an isolated DP receptor variant or a DP receptor variant over-expressed in a genetically engineered cell with a compound, and determining the level of an indicator which correlates with modulation of the DP receptor variant, where an alteration in the level of the indicator as compared to a control level indicates that the compound is a compound that modulates the DP receptor variant and where the DP receptor variant is a polypeptide containing an amino acid sequence having at least 50% amino acid identity with SEQ ID NO: 4, and the amino acid sequence of SEQ ID NO: 5 or 6 or a conservative variant of SEQ ID NO: 5 or 6.

Further provided herein is a method for identifying a compound that specifically binds to a DP receptor variant by contacting an isolated DP receptor variant or a DP receptor variant over-expressed in a genetically engineered cell with a compound, and determining specific binding of the compound to the DP receptor variant, where the DP receptor variant is a polypeptide containing an amino acid sequence having at least 50% amino acid identity with SEQ ID NO: 4, and the amino acid sequence of SEQ ID NO: 5 or 6 or a conservative variant of SEQ ID NO: 5 or 6.

The invention further provides a method for identifying a compound that differentially modulates a DP receptor variant by contacting an isolated DP receptor variant or a DP receptor variant over-expressed in a genetically engineered cell with a compound; determining the level of an indicator which correlates with modulation of the DP receptor variant; contacting a second receptor with the compound; determining the level of a corresponding indicator which correlates with modulation of the second receptor; and comparing the level of the indicator which correlates with modulation of the DP receptor variant with the level of the corresponding indicator which correlates with modulation of the second receptor, where a different level of the indicator which correlates with modulation of the DP receptor variant as compared to the level of the corresponding indicator indicates that the compound is a compound that differentially modulates the DP receptor variant and where the DP receptor variant is a polypeptide containing an amino acid sequence having at least 50% amino acid identity with SEQ ID NO: 4, and the amino acid sequence of SEQ ID NO: 5 or 6 or a conservative variant of SEQ ID NO: 5 or 6.

The present invention additionally provides a method for identifying a compound that differentially binds to a DP receptor variant by contacting an isolated DP receptor variant or a DP receptor variant over-expressed in a genetically engineered cell with a compound; determining specific binding of the compound to the DP receptor variant; contacting a second receptor with the compound; determining specific binding of the compound to the second receptor; and comparing the level of specific binding to the DP receptor variant with the level of specific binding to the second receptor, where a different level of specific binding to the DP receptor variant as compared to the level of specific binding to the second receptor indicates that the compound is a compound that differentially binds to a DP receptor variant and where the DP receptor variant is a polypeptide containing an amino acid sequence having at least 50% amino acid identity with SEQ ID NO: 4, and the amino acid sequence of SEQ ID NO: 5 or 6 or a conservative variant of SEQ ID NO: 5 or 6.

The methods of the invention can be useful for designing drugs that bind to or modulate the wild-type human DP receptor (SEQ ID NO: 4) in preference to an alternatively spliced DP receptor variant or for identifying compounds that bind to or modulate an alternatively spliced DP receptor variant in preference to other DP receptor variants or the wild type DP receptor. Compounds identified by a method of the invention can be therapeutically useful in preventing or reducing the severity of a condition where modulation of a DP receptor or a DP receptor variant is beneficial. Such conditions include, but are not limited to, pain; sleep regulation; treatment of ocular conditions such as glaucoma, various retinopathies and ocular hypertension; treatment of allergic disorders including allergic asthma and allergic rhinitis; and treatment of gastrointestinal disorders including, but not limited to, diarrhea, irritable bowel syndrome, Crohn's disease and other diseases involving gastrointestinal inflammation.

Prostaglandin D₂ has been implicated in a variety of physiological and pathophysiological processes. As an example, in the central nervous system, PGD₂ affects the sleep-wake cycle, acting as a potent endogenous sleep promoting substance in rats and other mammals including humans (Hayaishi, Soc. Lond. B. Biol. Sci. 2000:355:275-280 (2000)). PGD₂ also affects body temperature; olfactory function; hormone release and nociception; and is a potential intraocular pressure lowering agent for treatment of glaucoma. Additional roles for PGD₂, such as vasodilation and vasoconstriction and inhibition of platelet aggregation, may be specific to particular species (Giles et al., Br. J. Pharm. 96:291-300 (1989)).

Furthermore, PGD₂ is the major prostanoid produced in human mast cells upon immunological challenge, indicating that this molecule is an important mediator of allergic disorders such as allergic asthma and allergic rhinitis. PGD₂ is released in large amounts during asthmatic attacks in humans; in addition, DP receptor knockout mice do not develop asthmatic responses in an ovalbumin-induced model of asthma, further evidencing a role for this receptor in allergic disorders (Kobayashi and Narumiya, Prostaglandins & other Lipid Mediators 68-69:557-573 (2002)). These results indicate that DP variant modulators can be useful in treating asthma and allergic disorders as well as in treating pain, ocular conditions such as glaucoma, and in regulating sleep dysfunction.

The biologic effects of prostaglandin D₂ (PGD₂) are mediated through interaction with specific membrane-bound G protein-coupled prostanoid DP receptors, which have been cloned, for example, from mouse, rat and human tissues (Wright et al., supra, 1999; Oida et al., supra, 1997; Hirata et al., supra, 1994; and Boie et al., supra, 1995). The human DP receptor cDNA contains a 1080 bp open reading frame which encodes a 359 amino acid protein with a calculated molecular mass of 40,276. The human DP receptor contains three potential N-glycosylation sites, Asn-10, Asn-90 and Asn-297, in the amino-terminus and the first and third extracellular loops, respectively. There are also two potential protein kinase C phosphorylation sites, Ser-50 and Thr-145, located in the first and second cytoplasmic loops. Additional potential phosphorylation sites are positioned in the carboxy-terminal tail and may be important in receptor desensitization. Hydropathy analysis of the deduced amino acid sequence confirmed the presence of seven putative transmembrane domains, as expected for a G-protein coupled receptor (Boie et al., supra, 1995).

The least abundant of the prostanoid receptors, DP receptor mRNA is expressed at low levels in most tissues, and at high levels in leptomeninges, retina, and mucus-secreting cells of the gastrointestinal tract (Wright et al., Eur. J. Pharm. 377:101-115 (1999); Oida et al., FEBS Lett. 417:53-56 (1997); Hirata et al., Proc. Natl. Acad. Sci. USA 91:11192-11196 (1994); and Boie et al., J. Biol. Chem. 270: 18910-18916 (1995)). DP receptors also may be localized in platelets; neutrophils; non-chromaffin cells from adrenal medulla; smooth muscle cells from several tissues; and nerve cells including cells of the central nervous system.

The human DP receptor binds PGD₂ with a high-affinity binding site of 300 pM, and a lower-affinity site of about 13 nM. DP selective ligands include the agonist BW245C, and the partial agonist BWA868C (Coleman et al., Pharm. Rev. 46:205-229 (1994)). In cell culture, the DP receptor has an agonist order of affinity as follows: BW245C>PGD₂>>carbacyclin>PGE₂>>PGF2α=iloprost=U46619. Functionally, cloned DP receptors increase intracellular cAMP and Ca²⁺ (Hirata et al., supra, 1994; and Boie et al., supra, 1995), and may be regulated by phosphorylation.

The present invention relates to novel, alternatively spliced variants of the wild-type DP receptor such as the “DP VAR-1” variant disclosed in FIG. 2. Thus, the present invention provides an isolated polypeptide that contains an amino acid sequence having at least 50% amino acid identity with SEQ ID NO: 4, and the amino acid sequence of SEQ ID NO: 5 or 6 or a conservative variant of SEQ ID NO: 5 or 6. An isolated polypeptide of the invention can include, for example, an amino acid sequence having at least 80% amino acid identity with SEQ ID NO: 4, or an amino acid sequence having at least 90% amino acid identity with SEQ ID NO: 4. In one embodiment, the invention provides an isolated polypeptide that contains the amino acid sequence of SEQ ID NO: 2, or a conservative variant thereof. In other embodiments, the invention provides an isolated polypeptide containing the amino acid sequence of SEQ ID NO: 2, or consisting of SEQ ID NO: 2. Cells which contain any of the above exogenously expressed polypeptides also are provided by the invention.

The invention also provides an isolated nucleic acid molecule having a nucleotide sequence that encodes a polypeptide containing an amino acid sequence having at least 50% amino acid identity with SEQ ID NO: 4 and containing the amino acid sequence of SEQ ID NO: 5 or 6, or a conservative variant thereof. The invention also provides an isolated nucleic acid molecule having a nucleotide sequence that encodes a polypeptide containing an amino acid sequence having at least 80% or at least 90% amino acid identity with SEQ ID NO: 2 and containing the amino acid sequence of SEQ ID NO: 5 or 6, or a conservative variant thereof. The invention further provides an isolated nucleic acid molecule containing a nucleotide sequence that encodes the amino acid sequence of SEQ ID NO: 2, or a conservative variant thereof, such as the nucleotide sequence of SEQ ID NO: 1. The invention further provides a vector containing a nucleic acid molecule having a nucleotide sequence that encodes a polypeptide containing an amino acid sequence having at least 50% amino acid identity with SEQ ID NO: 4 and containing the amino acid sequence of SEQ ID NO: 5 or 6, or a conservative variant thereof; a vector containing a nucleic acid molecule having a nucleotide sequence that encodes a polypeptide containing the amino acid sequence of SEQ ID NO: 2, or a conservative variant thereof, and a vector containing a nucleic acid molecule having a nucleotide sequence that encodes the polypeptide of SEQ ID NO: 2. Host cells containing such vectors are further provided herein.

The invention relates, in part, to the identification of novel DP receptor variants. As used herein, the term DP receptor variant means a polypeptide containing an amino acid sequence that has at least 50% amino acid identity with the wild-type human DP receptor SEQ ID NO: 4 and further containing the amino acid sequence of SEQ ID NO: 5 or 6, or a conservative variant of SEQ ID NO: 5 or 6. A DP receptor variant can contain an amino acid sequence having, for example, at least 50% amino acid identity, at least 60% amino acid identity, at least 70% amino acid identity, at least 80% amino acid identity, at least 83% amino acid identity, at least 85% amino acid identity, at least 90% amino acid identity, at least 95% amino acid identity, or at least 98% amino acid identity with the wild-type human DP receptor SEQ ID NO: 4.

Based on the above, it is understood that species homologs of DP receptor variants that contain the amino acid sequence of SEQ ID NO: 5 or 6, or a conservative variant thereof, are encompassed by the definition of DP receptor variant as used herein. As non-limiting examples, an isolated polypeptide containing the amino acid sequence of SEQ ID NO: 2, or consisting of the amino acid sequence of SEQ ID NO: 2, is a DP receptor variant of the invention.

A DP receptor variant differs from the known wild-type human DP receptor polypeptide by containing the amino acid sequence of SEQ ID NO: 5 or 6, or a conservative variant of such an amino acid sequence. As used herein in reference to a specified amino acid sequence such as SEQ ID NO: 5 or 6, a conservative variant is a sequence in which a first amino acid is replaced by another amino acid or amino acid analog having at least one biochemical property similar to that of the first amino acid; similar properties include, yet are not limited to, similar size, charge, hydrophobicity or hydrogen-bonding capacity.

As an example, a conservative variant can be a sequence in which a first uncharged polar amino acid is conservatively substituted with a second (non-identical) uncharged polar amino acid such as cysteine, serine, threonine, tyrosine, glycine, glutamine or asparagine or an analog thereof. A conservative variant also can be a sequence in which a first basic amino acid is conservatively substituted with a second basic amino acid such as arginine, lysine, histidine, 5-hydroxylysine, N-methyllysine or an analog thereof. Similarly, a conservative variant can be a sequence in which a first hydrophobic amino acid is conservatively substituted with a second hydrophobic amino acid such as alanine, valine, leucine, isoleucine, proline, methionine, phenylalanine or tryptophan or an analog thereof. In the same way, a conservative variant can be a sequence in which a first acidic amino acid is conservatively substituted with a second acidic amino acid such as aspartic acid or glutamic acid or an analog thereof; a sequence in which an aromatic amino acid such as phenylalanine is conservatively substituted with a second aromatic amino acid or amino acid analog, for example, tyrosine; or a sequence in which a first relatively small amino acid such as alanine is substituted with a second relatively small amino acid or amino acid analog such as glycine or valine or an analog thereof. It is understood that a conservative variant of SEQ ID NO: 2, 5 or 6 can have one, two, three, four, five, ten, or more amino acid substitutions relative to the specified sequence and that such a conservative variant can include naturally and non-naturally occurring amino acid analogs.

It is understood that a fragment of a DP receptor variant containing the amino acid sequence of SEQ ID NO: 5 or 6 can be useful in a method of the invention. As non-limiting examples, a functional fragment of a DP receptor variant such as a ligand-binding fragment or a fragment of a DP receptor variant that is involved in signal transduction can be useful in a method of the invention in place of the full-length DP receptor variant. As further understood by one skilled in the art, a DP receptor variant can optionally include non-homologous amino acid sequence. As non-limiting examples, a DP receptor variant can contain an epitope tag or can be fused to a non-homologous polypeptide such as gluthionine S-transferase.

As discussed above, the DP receptor variant DP VAR-1 contains an amino acid sequence that is not present in the wild-type DP receptor SEQ ID NO: 4 (see FIG. 2). The alternatively spliced DP receptor variant DP VAR-1 contains unique carboxy terminal amino acid sequence disclosed herein as SEQ ID NO: 6. Furthermore, a ten amino acid sequence spanning the junction between conserved exon 1 and the additional exon A present in DP receptor variant DP VAR-1 is disclosed herein as SEQ ID NO: 5. The ten amino acid sequence begins with four amino acid residues that correspond to amino acid sequence present in conserved exon 1 and further includes six amino acid residues derived from exon A in DP receptor variant DP VAR-1. Thus, the invention also provides an isolated polypeptide containing the amino acid sequence of SEQ ID NO: 5 or 6. The invention further provides an isolated polypeptide containing an amino acid sequence having at least 50% amino acid identity with SEQ ID NO: 4 and containing the amino acid sequence of SEQ ID NO: 5 or 6, or a conservative variant thereof. As non-limiting examples, the invention provides an isolated polypeptide containing the amino acid sequence of SEQ ID NO: 2 or a conservative variant thereof, such as an isolated polypeptide containing or consisting of the amino acid sequence of SEQ ID NO: 2.

Further provided herein is an isolated polypeptide containing or consisting of substantially the same amino acid sequence as SEQ ID NO: 2. The term substantially the same, when used herein in reference to an amino acid sequence, means a polypeptide having a similar, non-identical sequence that is considered by those skilled in the art to be a functionally equivalent amino acid sequence. An amino acid sequence that is substantially the same as a reference amino acid sequence can have at least 70%, at least 80%, at least 90%, or at least 95% or more identity to the reference sequence. The term substantially the same amino acid sequence also includes sequences encompassing, for example, modified forms of naturally occurring amino acids such as D-stereoisomers, non-naturally occurring amino acids, amino acid analogs and mimetics, so long as the polypeptide containing such a sequence retains a functional activity of the reference DP receptor variant. A functional activity of a DP receptor variant of the invention can be, for example, the ability to bind a compound such as, but not limited to, BW245C or PGD₂, or the ability to increase intracellular cAMP or Ca²⁺ or to initiate a particular intracellular signal transduction pathway.

It is understood that minor modifications in primary amino acid sequence can result in a polypeptide that has a substantially equivalent function as compared to a polypeptide of the invention. These modifications can be deliberate, as through site-directed mutagenesis, or may be accidental such as through spontaneous mutation. For example, it is understood that only a portion of the entire primary structure of a DP receptor variant can be required in order to bind to compound such as BW245C or PGD₂. Moreover, fragments of a DP receptor variant of the invention containing the amino acid sequence of SEQ ID NO: 5 or 6 similarly are included within the definition of substantially the same amino acid sequence as long as at least one biological function of the DP receptor variant is retained. It is also understood that various molecules can be attached to a DP receptor variant or other polypeptide of the invention. These molecules include, without limitation, heterologous polypeptides, carbohydrates, lipids, or chemical moieties such as radioactive or fluorescent label moieties.

The invention further provides a DP receptor variant binding agent which binds the amino acid sequence of SEQ ID NO: 6, or an epitope thereof. As discussed above, SEQ ID NO: 6 represents the unique carboxy terminal amino acid sequence of alternatively spliced DP receptor variant DP VAR-1. A DP receptor variant binding agent of the invention can be, without limitation, an antibody or antigen binding fragment thereof which binds the amino acid sequence of SEQ ID NO: 6, or an epitope thereof.

As used herein, the term “DP receptor variant binding agent” means a molecule, such as a simple or complex organic molecule, carbohydrate, peptide, peptidomimetic, protein, glycoprotein, lipoprotein, lipid, nucleic acid molecule, antibody, aptamer or the like that specifically binds the unique DP receptor variant carboxy-terminal amino acid sequence disclosed herein as SEQ ID NO: 6, or an epitope thereof. It is understood that such a binding agent does not specifically bind to a wild-type DP receptor such as SEQ ID NO: 4 since a wild-type DP receptor does not contain the unique carboxy terminal amino acid sequence disclosed herein as SEQ ID NO: 6.

A DP receptor variant binding agent of the invention can be a polypeptide that specifically binds with high affinity or avidity to SEQ ID NO: 6, without substantial cross-reactivity to other unrelated sequences. The affinity of a DP receptor variant binding agent of the invention generally is greater than about 10⁻⁴ M and can be greater than about 10⁻⁶ M, typically being in the range of 10⁻⁴ M to 10⁻¹⁰ M. A DP receptor variant binding agent of the invention can bind, for example, with high affinity such as an affinity of 10⁻⁷ M to 10⁻¹⁰ M. Specific examples of binding agents of the invention include, but are not limited to, polyclonal and monoclonal antibodies that specifically bind an epitope within SEQ ID NO: 6; and nucleic acid molecules, nucleic acid analogs, and small organic molecules, identified, for example, by affinity screening of a nucleic acid or small molecule library against SEQ ID NO: 6. For certain applications, a DP receptor variant binding agent can be utilized that preferentially recognizes a particular conformational or post-translationally modified state of SEQ ID NO:6. It is understood that a DP receptor variant binding agent of the invention can be labeled with a detectable moiety, if desired, or rendered detectable by specific binding to a detectable secondary agent.

In one embodiment, a DP receptor variant binding agent of the invention is an antibody or antigen-binding fragment thereof. As used herein, the term “antibody” is used in its broadest sense to mean a polyclonal or monoclonal antibody or an antigen binding fragment of such an antibody. Such an antibody of the invention is characterized by having specific binding activity for SEQ ID NO: 6, or an epitope thereof, of at least about 1×10⁻⁵ M. Thus, Fab, F(ab′)₂, Fd and Fv fragments of an antibody, which retain specific binding activity for SEQ ID NO: 6, or an epitope thereof, are included within the definition of antibody as used herein. Specific binding activity can be readily determined by one skilled in the art, for example, by comparing the binding activity of the antibody to SEQ ID NO: 6, versus a control sequence. Methods of preparing polyclonal or monoclonal antibodies are well known to those skilled in the art. See, for example, Harlow and Lane, Antibodies: A Laboratory Manual, Cold Spring Harbor Laboratory Press (1988).

It is understood that the term antibody includes naturally occurring antibodies as well as non-naturally occurring antibodies such as, without limitation, single chain antibodies, chimeric, bi-functional and humanized antibodies, and antigen-binding fragments thereof. Such non-naturally occurring antibodies can be constructed using solid phase peptide synthesis, produced recombinantly or obtained, for example, by screening combinatorial libraries consisting of variable heavy chains and variable light chains as described in Huse et al., Science 246:1275-1281 (1989). These and other methods of making, for example, chimeric, humanized, CDR-grafted, single chain, and bi-functional antibodies are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); and Borrabeck, Antibody Engineering, 2d ed. (Oxford University Press 1995)).

An antibody of the invention can be prepared using as an antigen a polypeptide or peptide containing SEQ ID NO: 6, or an epitope thereof, which can be prepared, for example, from natural sources, produced recombinantly, or chemically synthesized. Such a polypeptide or peptide is a functional antigen if the polypeptide or peptide can be used to generate an antibody that specifically binds SEQ ID NO: 6, or an epitope thereof. As is well known in the art, a non-antigenic or weakly antigenic polypeptide or peptide can be made antigenic by coupling the polypeptide or peptide to a carrier molecule such as bovine serum albumin (BSA) or keyhole limpet hemocyanin (KLH). Various other carrier molecules and methods for coupling a polypeptide or peptide to a carrier molecule are well known in the art (see, for example, Harlow and Lane, supra, 1988). An antigenic polypeptide or peptide can also be generated by expressing the polypeptide or peptide as a fusion protein, for example, fused to glutathione S transferase, polyHis or the like. Methods for expressing polypeptide fusions are well known to those skilled in the art as described, for example, in Ausubel et al., Current Protocols in Molecular Biology (Supplement 47), John Wiley & Sons, New York (1999).

The present invention also provides a cell that includes an exogenously expressed polypeptide containing the amino acid sequence of SEQ ID NO: 5 or 6. Further provided herein is a cell that includes an exogenously expressed polypeptide containing an amino acid sequence having at least 50% amino acid identity with SEQ ID NO: 2, and containing the amino acid sequence of SEQ ID NO: 5 or 6, or a conservative variant thereof. The invention provides, for example, a cell that includes an exogenously expressed polypeptide containing the amino acid sequence of SEQ ID NO: 2, or a conservative variant thereof.

Such a cell containing an exogenously expressed polypeptide of the invention can be generated by expressing a nucleic acid molecule encoding the polypeptide in a suitable host cell, such as a bacterial cell, yeast cell, insect cell, oocyte or other amphibian cell, or mammalian cell, using methods well known in the art. Suitable expression vectors are well known in the art and include vectors in which a nucleic acid molecule is operatively linked to a regulatory element such as a promoter or enhancer region that is capable of regulating expression of a linked nucleic acid molecule. Appropriate expression vectors include, without limitation, those that can be replicated in eukaryotic or prokaryotic cells, those that remain episomal as well as those which integrate into the host cell genome, and those including constitutive, inducible or regulated promoters, enhancers or other regulatory elements.

Suitable expression vectors for prokaryotic or eukaryotic cells are well known to those skilled in the art (see, for example, Ausubel et al., supra, 1999). Eukaryotic expression vectors can contain, for example, a regulatory element such as, but not limited to, the SV40 early promoter, the cytomegalovirus (CMV) promoter, the mouse mammary tumor virus (MMTV) steroid-inducible promoter, the Moloney murine leukemia virus (MMLV) promoter, and the like. One skilled in the art will know or can readily determine an appropriate expression vector for a particular host cell.

Useful expression vectors optionally contain a regulatory element that provides cell or tissue specific expression or inducible expression of the operatively linked nucleic acid molecule. One skilled in the art can readily determine an appropriate tissue-specific promoter or enhancer that allows expression of a polypeptide of the invention in a desired tissue. Furthermore, any of a variety of inducible promoters or enhancers can also be included in an expression vector for regulated expression of a polypeptide of the invention. Such inducible systems include, yet are not limited to, a tetracycline inducible gene regulatory region (Gossen & Bijard, Proc. Natl. Acad. Sci. USA 89:5547-5551 (1992); Gossen et al., Science 268:1766-1769 (1995); Clontech, Palo Alto, Calif.); a metallothionein promoter inducible by heavy metals; an insect steroid hormone responsive gene regulatory region responsive to ecdysone or related steroids such as muristerone (No et al., Proc. Natl. Acad. Sci. USA 93:3346-3351 (1996); Yao et al., Nature 366:476-479 (1993); Invitrogen, Carlsbad, Calif.); a mouse mammary tumor virus (MMTV) gene regulatory region induced by steroids such as glucocortocoid and estrogen (Lee et al., Nature 294:228-232 (1981); and a heat shock promoter.

An expression vector useful in the invention can be a viral vector such as, without limitation, a retrovirus, adenovirus, adeno-associated virus, lentivirus, or herpesvirus vector. Viral based systems provide the advantage of being able to introduce relatively high levels of a heterologous nucleic acid molecule into a variety of cells. Additionally, certain viral vectors can introduce heterologous DNA into non-dividing cells. A variety of suitable viral expression vectors are well known in the art and include, without limitation, herpes simplex virus vectors (U.S. Pat. No. 5,501,979), vaccinia virus vectors (U.S. Pat. No. 5,506,138), cytomegalovirus vectors (U.S. Pat. No. 5,561,063), modified Moloney murine leukemia virus vectors (U.S. Pat. No. 5,693,508), adenovirus vectors (U.S. Pat. Nos. 5,700,470 and 5,731,172), adeno-associated virus vectors (U.S. Pat. No. 5,604,090), constitutive and regulatable retrovirus vectors (U.S. Pat. Nos. 4,405,712; 4,650,764 and 5,739,018, respectively), papilloma virus vectors (U.S. Pat. Nos. 5,674,703 and 5,719,054), and the like.

A cell can be generated that transiently or stably expresses an exogenously expressed polypeptide of the invention. Expression vectors for transient or stable expression of a polypeptide of the invention can be introduced into cells using transfection methods well known to one skilled in the art. Such methods include, without limitation, infection using viral vectors, lipofection, electroporation, particle bombardment and transfection such as calcium-phosphate mediated transfection. Detailed procedures for these methods can be found in Sambrook et al., Molecular Cloning: A Laboratory Manual Cold Spring Harbor Laboratory Press (1989), and the references cited therein. Useful mammalian expression vectors and methods of introducing such vectors into mammalian cells either ex vivo or in vivo are well known in the art. As non-limiting examples, a plasmid expression vector can be introduced into a cell by calcium-phosphate mediated transfection, DEAE dextran-mediated transfection, lipofection, polybrene- or polylysine-mediated transfection, electroporation, or by conjugation to an antibody, gramacidin S, artificial viral envelope or other intracellular carrier. A viral expression vector can be introduced into a cell by infection or transduction, for example, or by encapsulation in a liposome. It further is understood that polypeptides can be delivered directly into cells using a lipid-mediated delivery system (Zelphati et al., J. Biol. Chem. 276:35103-35110 (2001)) to produce a cell that contains an exogenously expressed polypeptide of the invention.

Exemplary host cells that can be used to exogenously express a polypeptide of the invention include, yet are not limited to, mammalian primary cells; established mammalian cell lines such as COS, CHO, HeLa, NIH3T3, HEK 293, and HEK 293/EBNA cells; amphibian cells such as Xenopus embryos and oocytes; and other vertebrate cells. Exemplary host cells further include, without limitation, insect cells such as Drosophila, Spodoptera frugiperda and other cells compatible with baculovirus expression systems (Murakimi et al., Cytokine, 13:18-24 (2001)); yeast cells such as Saccharomyces cerevisiae, Saccharomyces pombe, or Pichia pastoris; and prokaryotic cells such as Escherichia coli. Following transfection, cells exogenously expressing a polypeptide of the invention can be selected, for example, using drug resistance. A quantitative assay such as, for example, immunoblot analysis, immunoprecipitation or ELISA can determine the amount of a polypeptide of the invention expressed in a transfected cell. Such methods are known to one skilled in the art and can be found, for example, in Ausubel et al., supra, 1989, or in Harlow et al., supra, 1988.

Further provided herein are methods for identifying a compound that modulates a DP receptor variant, identifying a compound that differentially modulates a DP receptor variant, identifying a compound that specifically binds a DP receptor variant, and identifying a compound that differentially binds to a DP receptor variant. In particular, the invention provides a method for identifying a compound that modulates a DP receptor variant by contacting a DP receptor variant with a compound and determining the level of an indicator which correlates with modulation of a DP receptor variant, where an alteration in the level of the indicator as compared to a control level indicates that the compound is a compound that modulates the DP receptor variant. Further provided herein are methods for identifying a compound that modulates a DP receptor variant by contacting an isolated DP receptor variant or a DP receptor variant over-expressed in a genetically engineered cell with a compound and determining the level of an indicator which correlates with modulation of a DP receptor variant, where an alteration in the level of the indicator as compared to a control level indicates that the compound is a compound that modulates the DP receptor variant.

As used herein in reference to a DP receptor variant, the term “modulates” means the ability to alter a characteristic of a DP receptor variant. A characteristic of a DP receptor variant that can be altered can include, without limitation, an amount, activity, or physical conformation of a DP receptor variant. As a non-limiting example, a compound that modulates a DP receptor variant can increase or decrease the binding of a DP receptor variant to a ligand such as, without limitation, BW245C, PGD₂, ZK118.132 and L-644,698. Also, for example, a compound can increase or decrease the binding of a DP receptor variant to an intracellular signaling molecule that initiates a signal transduction pathway within a cell. It is understood that compounds that modulate a DP receptor variant encompass compounds that specifically bind to a DP receptor variant as well as compounds that bind non-specifically to a DP receptor variant including, without limitation, those described in Breyer et al., Ann. Rev. Pharm. Toxicol. 41:661-690 (2001), U.S. Pat. No. 5,093,329; Pons et al., Eur. J. Pharm. 261:237-247 (1994); and Hamish Wright et al., Br. J. Pharm. 123:1317-1324 (1998).

A method of the invention for identifying a compound that modulates a DP receptor variant involves determining the level of an indicator which correlates with modulation of a DP receptor variant, where an alteration in the level of the indicator as compared to a control level indicates that the compound modulates the DP receptor variant. As used herein, the term indicator means a detectable substance which is altered qualitatively or quantitatively in response to modulation of a DP receptor variant. An indicator can be a substance that is normally present in a cell such as a signal transduction molecule, or a substance that is exogenously expressed or otherwise added to a cell, the level of which correlates with modulation of a DP receptor variant. One example of an indicator is luciferase. Signal transduction molecules are intracellular substances such as, without limitation, cyclic AMP, inositol phosphates and calcium, the level of which can be altered in response to modulation of a DP receptor variant.

As understood by those of skill in the art, assay methods for identifying compounds that modulate a DP receptor variant generally require comparison to a control. For example, in a method of the invention an alteration in the level of an indicator which correlates with modulation of a DP receptor variant is compared to a control level of the indicator. One type of a control is a sample that is treated substantially the same as the DP receptor variant which is contacted with a compound, with the distinction that the control sample is not exposed to the compound. Controls include, but are not limited to, historical reference values, and samples that are assayed simultaneously or sequentially in comparison to the DP receptor variant which is contacted with the compound.

In one embodiment, a method of the invention is practiced using calcium as the indicator. For example, as disclosed herein in Example III, a FLIPR assay can be used to identify compounds that modulate a DP receptor variant by determining the level of calcium that results after contacting a receptor with a compound. Exogenously expressed substances such as, for example, luciferase, β-galactosidase and green fluorescent protein (GFP) also can be indicators useful in a method of the invention (see Example III).

Further provided herein are methods for identifying a compound that specifically binds to a DP receptor variant by contacting a DP receptor variant with a compound and determining specific binding of the compound to the DP receptor variant. Additionally provided herein are methods for identifying a compound that specifically binds to a DP receptor variant by contacting an isolated DP receptor variant or a DP receptor variant over-expressed in a genetically engineered cell with a compound and determining specific binding of the compound to the DP receptor variant.

As used herein in reference to a compound and a DP receptor variant, the term “specific binding” means binding with an affinity for the target DP receptor variant that is measurably higher than the affinity for an unrelated polypeptide such as an unrelated G protein coupled receptor such as a rhodopsin receptor. For example, a polypeptide or small molecule compound that specifically binds a DP receptor variant has an affinity for the DP receptor variant that is measurably higher than its affinity for an unrelated polypeptide. Binding affinity can be low or high so long as the binding is sufficient to be detectable. For example, a compound can specifically bind a DP receptor variant with a binding affinity (Kd) of about 10⁻⁴ M or less, 10⁻⁵ M or less, 10⁻⁶ M or less, 10⁻⁷ M or less, 10⁻⁸ M or less, or 10⁻⁹ M or less. Several methods for detecting or measuring specific binding are well known in the art and discussed further below.

The screening methods of the invention can be practiced, for example, using a DP receptor variant over-expressed in a genetically engineered cell. As used herein, the term genetically engineered cell means a cell having genetic material which is altered by the hand of man. Such a cell can contain a transient or permanent alteration of its genetic material including, for example, alteration in genomic or episomal genetic material. The genetic material in a genetically engineered cell can be altered using, without limitation, an exogenously expressed nucleic acid molecule, chemical mutagen or transposable element. It is understood that a genetically engineered cell can contain one or more man-made alterations, for example, a cell can be co-transfected with more than one expression vector. As used herein in relation to a DP receptor variant in a genetically engineered cell, the term “over-expressed” means having a protein level of a DP receptor variant greater than the level seen in a corresponding non-genetically engineered cell.

As understood by one skilled in the art, a DP receptor variant can be over-expressed in a genetically engineered cell, for example, by exogenously expressing a nucleic acid molecule encoding the DP receptor variant in a cell as described herein above. It is further understood that a DP receptor variant can be over-expressed in a cell that does not normally express the DP receptor variant, or in a cell that naturally expresses the endogenous DP receptor variant. As a non-limiting example, a DP receptor variant can be over-expressed in a cell that endogenously expresses the DP receptor variant at a low level. In addition, a DP receptor variant can be over-expressed in a genetically engineered cell, for example, by expressing a regulatory molecule in the cell to increase expression of the endogenous DP receptor variant. Another example of a method whereby a DP receptor variant can be over-expressed in a genetically engineered cell is recombination of a heterologous regulatory region such as, without limitation, a promoter, enhancer or 3′ regulator, in the cell such that the heterologous regulatory region results in over-expression of endogenous DP receptor variant. As understood by one skilled in the art, over-expression of a DP receptor variant in a genetically engineered cell includes, without limitation, over-expression of the variant on the surface of the cell, within a cell membrane or in the cytosolic portion of the cell.

A DP receptor variant also can be over-expressed in a cell using a chemical agent. Thus, the invention provides a method for identifying a compound that modulates a DP receptor variant by contacting the DP receptor variant with a compound, where the DP receptor variant is over-expressed in a cell using a chemical agent, and determining the level of an indicator which correlates with modulation of a DP receptor variant, where an alteration in the level of the indicator as compared to a control level indicates that the compound is a compound that modulates the DP receptor variant. The invention also provides a method for identifying a compound that specifically binds to a DP receptor variant by contacting the DP receptor variant with a compound, where the DP receptor variant is over-expressed in a cell using a chemical agent, and determining specific binding of the compound to the DP receptor variant. Chemical agents that can result in over-expression of a DP receptor variant can include, without limitation, chemicals that induce the level or activity of regulatory factor, such as a transcription factor, that is involved in DP receptor variant expression.

As described above, the methods of the invention can be practiced with a cell that over-expresses a DP receptor variant. In addition, it is understood that an extract of a cell that over-expresses a DP receptor variant, such as a genetically engineered cell that over-expresses a DP receptor variant, can be useful in the methods of the invention. Methods for generating different types of cellular extracts including, without limitation, whole cell extracts, membrane extracts, cytosolic extracts and nuclear extracts are well known in the art. As a non-limiting example, receptor enriched plasma membrane fractions can be obtained by continuous or discontinuous gradients of, for example, sucrose as described in Woodward and Lawrence, Biochemical Pharmacology 47:1567-1674 (1994).

Isolated DP receptor variants also can be useful in the screening methods of the invention. As used herein in reference to a DP receptor variant, the term “isolated” means the DP receptor variant is substantially separated from other polypeptides. For example, an isolated DP receptor variant derived from a cell can be substantially purified away from other polypeptides in the cell. An isolated DP receptor variant can contain non-polypeptide components, for example, an isolated DP receptor variant can be associated with a natural or artificial lipid containing membrane. In one embodiment, a method of the invention is practiced with an isolated DP receptor variant that contains an amino acid sequence having at least 50% amino acid identity with SEQ ID NO: 4 and containing the amino acid sequence of SEQ ID NO: 5 or 6, or a conservative variant thereof. In another embodiment, a method of the invention is practiced with an isolated DP receptor variant that contains an amino acid sequence having at least 80% or at least 90% amino acid identity with SEQ ID NO: 4 and containing the amino acid sequence of SEQ ID NO: 5 or 6, or a conservative variant thereof. In a further embodiment, a method of the invention is practiced with a DP receptor variant that contains the amino acid sequence of SEQ ID NO: 2, or a conservative variant thereof. In a yet further embodiment, a method of the invention is practiced with an isolated DP receptor variant that contains or consists of SEQ ID NO: 2.

A DP receptor variant of the invention can be prepared in isolated form using conventional biochemical purification methods, starting either from tissues containing the desired DP receptor variant or from recombinant sources. A DP receptor variant can be isolated by any of a variety of methods well-known in the art, including, but not limited to, precipitation, gel filtration, ion-exchange, reverse-phase and affinity chromatography, and combinations thereof. Other well-known methods for protein isolation are described in Deutscher et al., Guide to Protein Purification: Methods in Enzymology Vol. 182 (Academic Press, (1990)). Methods suitable for isolating a DP receptor variant of the invention using biochemical purification are known in the art as described for example, in Venter and Harrison, (eds), Receptor Purification Procedures ( Liss, (1984)); Litwack, Receptor Purification: Receptors for CNS Agents, Growth Factors, Hormones, & Related Substances, (Humana Press, (1990)); or Litwack, Receptor Purification: Receptors for Steroid Hormones, Thyroid Hormones, Water Balancing Hormone, & Others (Humana Press, (1990)). Purification of the receptor variant can be routinely monitored, for example, by an immunological assay or functional assay such as a ligand binding assay.

An isolated DP receptor variant of the invention also can be produced by chemical synthesis. As a non-limiting example, synthetic isolated DP receptor variants, including fragments thereof, can be produced using an Applied Biosystems, Inc. Model 430A or 431A automatic peptide synthesizer (Foster City, Calif.) employing the chemistry provided by the manufacturer. Methods for synthesizing isolated polypeptides are well known in the art (see, for example, Bodanzsky, Principles of Peptide Synthesis (1st ed. & 2d rev. ed.), Springer-Verlag, New York, N.Y. (1984 & 1993), see Chapter 7; Stewart and Young, Solid Phase Peptide Synthesis, (2d ed.), Pierce Chemical Co., Rockford, Ill. (1984)).

In the methods of the invention for identifying a compound that modulates, or specifically binds to, a DP receptor variant, an isolated DP receptor variant or DP receptor variant over-expressed in a genetically engineered cell can be contacted with a compound in a solution under conditions suitable for interaction between the DP receptor variant and compound. Such contact can occur in vitro, such as in an isolated cell in cell culture, in a whole or partially purified cell extract, or with an isolated polypeptide. As used herein, the term “in vitro” means in an artificial environment outside of a living organism or cell. Assays performed in a test tube, microcentrifuge tube, 96 well plate, 384 well plate, 1536 well plate or other assay format outside of an organism or living cell are in vitro assays. Experiments performed in cells or tissues that have been fixed and are therefore dead (sometimes referred to as in situ experiments) or using cell-free extracts from cells are in vitro. Contact can also occur in vivo using, for example, isolated living cells, living tissues or whole animals.

Conditions suitable for contacting an isolated DP receptor variant or DP receptor variant over-expressed in a genetically engineered cell with a compound are dependent on the characteristics of the DP receptor variant and the compound. For example, the overall charge of the DP receptor variant and the compound can be considered when adjusting the salt concentration or pH of a buffering solution to optimize the specific binding or modulation of the DP receptor variant by the compound. Usually a salt concentration and pH in the physiological range, for example, about 100 mM KCl and pH 7.0 are reasonable starting points. In addition, other components such as glycerol or protease inhibitors can be added to the solution, for example, to inhibit polypeptide degradation. It is understood that the stability of the contact between the DP receptor variant and the compound can be effected by the temperature at which such contact occurs and that the optimal temperature for contact can be routinely determined by those skilled in the art. For example, reactions can be performed on ice (4° C.), at room temperature (about 25° C.) or at body temperature (37° C.). Suitable conditions can be similar or identical to conditions used for binding of a compound to the wild-type human DP receptor. Such conditions are known in the art and include, for example, contact in a binding buffer containing 10 mM BES/KOH (pH 7.0) with 1 mM EDTA, 10 mM MnCl₂, 0.8 nM [³H]PGD₂ and 60 ug of protein from the 100,000×g membrane fraction as described in Boie et al., supra, 1995.

The screening methods of the invention are useful for identifying compounds that modulate or differentially modulate, or that specifically or differentially bind a DP receptor variant. As used herein, the term “compound” means a molecule of natural or synthetic origin. A compound can be, without limitation, a small organic or inorganic molecule, polypeptide, peptide, peptidomimetic, non-peptidyl compound, carbohydrate, lipid, antibody or antibody fragment, aptamer, or nucleic acid molecule. In one embodiment, the compound is a small organic molecule. It is understood that a compound can have a known or unknown structure, and can be assayed as an isolated molecule or as part of a population of compounds such as a library.

As understood by one skilled in the art, a compound can specifically bind to a DP receptor variant without modulating the DP receptor variant; specifically bind to a DP receptor variant, thereby modulating the DP receptor variant; or modulate a DP receptor variant without specifically binding the DP receptor variant. Compounds that specifically bind to a DP receptor variant can include, without limitation, BW245C, PGD₂; ZK118.132; L-644,698; prostanoid-like compounds; and non-prostanoid-like structures identified as DP receptor ligands, for example, by screening of chemical libraries. A compound that modulates a DP receptor variant but does not directly bind to the DP receptor variant can be, for example, a compound that binds to or effects the activity of a polypeptide in a cell, where that polypeptide increases or decreases the level of a DP receptor variant. Such polypeptides include, without limitation, transcription or translation regulatory factors, signal transduction polypeptides; kinases and phosphatases; polypeptides that bind to a DP receptor variant; and anti-sense oligonucleotides, inhibitor RNA molecules and ribozymes, which act on the nucleic acid that encodes the DP receptor variant.

Compounds that modulate or specifically bind to a DP receptor variant further include, but are not limited to, agonists and antagonists. An agonist can be a compound that binds to a receptor and activates it, producing a pharmacological response such as contraction, relaxation, secretion, or enzyme activation. An antagonist is a compound which can attenuate the effect of an agonist. An antagonist can be competitive, meaning it binds reversibly to a region of the receptor in common with an agonist, but occupies the site without activating the effector mechanism. The effects of a competitive antagonist can be overcome by increasing the concentration of agonist, thereby shifting the equilibrium and increasing the proportion of receptors occupied by agonist. Alternatively, antagonists can be non-competitive, where no amount of agonist can completely overcome the inhibition once it has been established. Non-competitive antagonists can bind covalently to the agonist binding site (called competitive irreversible antagonists), in which case there is a period before the covalent bond forms during which competing ligands can prevent the inhibition. Other types of non-competitive antagonists act allosterically at a different site on the receptor.

Other classes of compounds that can modulate or specifically bind to a DP receptor variant include inverse agonists, which are compounds which produce an opposite physiological effect to that of an agonist, yet act at the same receptor. Such compounds have also been described as negative antagonists, or as having negative efficacy. Another class of compounds that can modulate or specifically bind to a DP receptor variant is partial agonists, which are agonists that are unable to produce maximal activation of the receptor.

A library of compounds can be useful in the screening methods of the invention. Such a library can be a random collection of compounds or a focused collection of compounds, for example, compounds that are rationally designed or pre-selected based on physical or functional characteristics. For example, a library of prostanoids or prostanoid-related compounds can be useful in the screening methods of the invention. Libraries useful in the methods of the invention include, yet are not limited to, natural product libraries derived from, without limitation, microorganisms, animals, plants, and marine organisms; combinatorial chemical or other chemical libraries such as those containing randomly synthesized compounds; combinatorial libraries containing structural analogs of prostanoids or other known compounds, or random or biased assortments of, for example, small organic molecules, polypeptides, oligonucleotides, and combinations thereof. Still other libraries of interest include peptidomimetic, multiparallel synthetic collection, and recombinatorial libraries. Combinatorial and other chemical libraries are known in the art, as described, for example, in Myers, Curr. Opin. Biotechnol. 8:701-707 (1997). Appropriate libraries can be assembled from catalog sources such as Cayman Chemical Co. (Ann Arbor, Mich.), BIOMOL Research Laboratories, Inc. (Plymouth Meeting, Pa.), Tocris Cooksoon Inc. (Ellisville, Mo.), and others. These libraries can include, without limitation, fatty acids, fatty acid amides and esters, and eicosanoids.

In a screening method of the invention, the members of a library of compounds can be assayed for activity individually, in pools, or en masse. An example of en masse screening to identify a compound that modulates or specifically binds to a DP receptor variant is as follows: a library of compounds is assayed in pools for the ability to modulate or specifically bind a DP receptor variant; the sub-population which modulates or specifically binds the DP receptor variant is subdivided; and the assay is repeated as needed in order to isolate an individual compound or compounds from the library that modulate or specifically bind the DP receptor variant.

The methods of the invention can utilize high throughput screening (HTS) techniques to identify compounds that modulate or specifically bind to a DP receptor variant. HTS assays permit screening of large numbers of compounds in an efficient manner. Cell-based high throughput screening systems include, but are not limited to, melanophore assays, yeast-based assay systems, and mammalian cell expression systems (Jayawickreme and Kost, Curr. Opin. Biotechnol. 8:629-634 (1997)). Automated and miniaturized high throughput screening assays are also useful in the methods of the invention (Houston and Banks, Curr. Opin. Biotechnol. 8:734-740 (1997)). High throughput screening assays are designed to identify “hits” or “lead compounds” having the desired modulating or specific binding activity, from which modified compounds can be prepared to improve a property of the initial lead compound. Chemical modification of the “hit” or “lead compound” can be based on an identifiable structure/activity relationship (SAR) between the “hit” and a DP receptor variant of the invention. It is understood that assays such as the melanophore and radioligand binding assays disclosed below, and the FLIPR and luciferase assays disclosed in Example III, can be performed as conventional or high through-put screening assays to identify a compound that modulates or specifically binds to a DP receptor variant, according to a method of the invention.

Various types of assays can be useful for identifying a compound that modulates or specifically binds to a DP receptor variant in a method of the invention. For example, several assays can be used to measure specific binding of a compound to a DP receptor variant in a method of the invention. A classic assay used for measuring specific binding of a compound to a receptor is a radioligand binding assay. Radioligand binding assays can be performed on cells or in solution, for example, using isolated cell membranes. As a non-limiting example, cells or cell membranes that transiently or stably over-express a DP receptor variant can be incubated with a ligand including a novel or known ligand such as radioactively labeled PGD₂. After washing away any unbound radioactively labeled PGD₂, compounds of interest can be incubated with the cells. After incubation, the solution around the cells is collected and the amount of radioactively labeled PGD₂ in the solution is determined using, for example, a scintillation counter. Compounds that specifically bind to the DP receptor variant displace radioactively labeled PGD₂ from the receptor and thereby increase radioactively labeled PGD₂ in the solution. A method for a radioligand binding assay using PGD₂ and membranes prepared from cells transiently transfected with DP receptor is described, for example, in Boie et al., J. Biol. Chem. 270:18910-18916 (1995). As understood by one skilled in the art, a ligand such as PGD₂ also can be labeled with a non-radioactive moiety such as a fluorescent moiety.

A variety of other assays well known in the art can be used to determine specific binding of a compound to a DP receptor variant in a method of the invention. Such assays include, without limitation, detecting specific binding of a labeled compound to a DP receptor variant which is immobilized. For example, a compound can be conjugated to a radiolabel, fluorescent label or enzyme label such as alkaline phosphatase, horse radish peroxidase or luciferase. Labeled compound can then bind to a DP receptor variant, for example a DP receptor variant membrane preparation, which is immobilized, for example, on a solid support such as a latex bead. Unbound compound can be washed away, and the amount of specifically bound compound can be detected based on its label. Fluorescently labeled compound can also be bound to a DP receptor variant in solution and bound complexes detected, for example, using a fluorescence polarization assay (Degterev et al., Nature Cell Biology 3:173-182 (2001)). Such assays also can be performed where the DP receptor variant is labeled and the compound is immobilized or in solution. One skilled in the art understands that a variety of additional means can be used to determine specific binding to a DP receptor variant; as non-limiting examples, binding of a compound to a ¹⁵N-labeled DP receptor variant can be detected using nuclear magnetic resonance (NMR), or specific binding can be determined using an antibody that specifically recognizes a ligand-bound DP receptor variant.

High-throughput assays for determining specific binding to a DP receptor variant further include, but are not limited to, scintillation proximity assays (Alouani, Methods Mol. Biol. 138:135-41 (2000)). Scintillation proximity assays involve the use of a fluomicrosphere coated with an acceptor molecule, such as an antibody, to which an antigen will bind selectively in a reversible manner. For example, a compound can be bound to a fluomicrosphere using an antibody that specifically binds to the compound, and contacted with a labeled DP receptor variant. If the labeled DP receptor variant specifically binds to the compound, the radiation energy from the labeled DP receptor variant is absorbed by the fluomicrosphere, thereby producing light which is easily measured. Such assays can also be performed where the DP receptor variant is bound to the fluomicrosphere, and the compound is labeled.

Additional assays suitable for determining specific binding of a compound to a DP receptor variant in a screening method of the invention include, without limitation, UV and chemical cross-linking assays (Fancy, Curr. Opin. Chem. Biol. 4:28-33 (2000)) and biomolecular interaction analyses (Weinberger et al., Pharmacogenomics 1:395-416 (2000)). Specific binding of a compound to a DP receptor variant can be determined by cross-linking these two components, if they are in contact with each other, using UV or a chemical cross-linking agent. In addition, a biomolecular interaction analysis (BIA) can detect whether two components are in contact with each other. In such an assay, one component, such as a DP receptor variant (for example, a membrane preparation containing a DP receptor variant) is bound to a BIA chip, and a second component such as a compound is passed over the chip. If the two components specifically bind, the contact results in an electrical signal which is readily detected.

In addition, virtual computational methods and the like can be used to identify compounds that modulate or specifically bind to a DP receptor variant in a screening method of the invention. Exemplary virtual computational methodology involves virtual docking of small-molecule compounds on a virtual representation of a DP receptor variant structure in order to determine or predict specific binding. See, for example, Shukur et al., supra, 1996; Lengauer et al., Current Opinions in Structural Biology 6:402-406 (1996); Choichet et al., Journal of Molecular Biology 221:327-346 (1991); Cherfils et al., Proteins 11:271-280 (1991); Palma et al., Proteins 39:372-384 (2000); Eckert et al., Cell 99:103-115 (1999); Loo et al., Med. Res. Rev. 19:307-319 (1999); and Kramer et al., J. Biol. Chem. (2000).

One type of assay that does not directly measure binding to a DP receptor variant, but measures activation of a signal transduction pathway, is an assay based on melanophores, which are skin cells that provide pigmentation to an organism (Lerner, Trends Neurosci. 17:142-146 (1994)). In numerous animals, including fish, lizards and amphibians, melanophores are used, for example, for camouflage. The color of the melanophore is dependent on the intracellular position of melanin-containing organelles, termed melanosomes. Melanosomes move along a microtubule network and are clustered to give a light color or dispersed to give a dark color. The distribution of melanosomes is regulated by G protein coupled receptors and cellular signaling events, where increased concentrations of second messengers such as cyclic AMP and diacylglycerol result in melanosome dispersion and darkening of melanophores. Conversely, decreased concentrations of cyclic AMP and diacylglycerol result in melanosome aggregation and lightening of melanophores.

A melanophore-based assay can be advantageously used to identify a compound that modulates or specifically binds to a DP receptor variant, due to the regulation of melanosome distribution by DP receptor variant-stimulated intracellular signaling. For example, a DP receptor variant can be over-expressed in genetically engineered melanophore cells, for example, frog melanophore cells. Compounds that modulate or specifically bind to the DP receptor variant can stimulate or inhibit G protein coupled receptor signaling. Both stimulation or inhibition of signaling can be determined since the system can be used to detect both aggregation of melanosomes and lightening of cells, and dispersion of melanosomes and darkening of cells. Thus, the color of the cells, determined by the level of melanin in the cells, is an indicator that can be used to identify a compound that modulates or specifically binds to a DP receptor variant in a method of the invention.

In addition to the methods described above for identifying a compound that modulates or specifically binds a DP receptor variant, the invention also provides related methods for identifying a compound that differentially modulates or differentially binds to a DP receptor variant. It is understood that the DP receptor variants, cells, compounds, indicators, conditions for contacting, and assays, described above also can be applied to methods for identifying a compound that differentially modulates or differentially binds to a DP receptor variant.

Provided herein is a method for identifying a compound that differentially modulates a DP receptor variant by a) contacting an isolated DP receptor variant or a DP receptor variant over-expressed in a genetically engineered cell with a compound; b) determining the level of an indicator which correlates with modulation of a DP receptor variant; c) contacting a second receptor with the compound; d) determining the level of a corresponding indicator after contacting of the compound to the second receptor; and e) comparing the level of the indicator from step (b) with the level of the corresponding indicator from step (d), where a different level of the indicator from step (b) compared to the level of the corresponding indicator from step (d) indicates that the compound is a compound that differentially modulates the DP receptor variant, and where the DP receptor variant contains an amino acid sequence having at least 50% amino acid identity with SEQ ID NO: 4 and contains the amino acid sequence of SEQ ID NO: 5 or 6, or a conservative variant thereof.

As described above, an indicator is a detectable substance which is altered qualitatively or quantitatively in response to modulation of a DP receptor variant. A “corresponding indicator” is an indicator that can be compared to the indicator which correlates with modulation of the DP receptor variant in step (b). For example, a corresponding indicator can be the same indicator as the indicator which correlates with modulation of the DP receptor variant in step (b). In addition, for example, a corresponding indicator can be a different indicator as the indicator which correlates with modulation of the DP receptor variant in step (b) so long as the corresponding indicator can be compared to the indicator which correlates with modulation of the DP receptor variant in step (b). As a non-limiting example, the indicator in step (b) can be calcium, and the corresponding indicator can be a substance whose amount is directly correlated with calcium level, such as a signal transduction molecule. As a further non-limiting example, the indicator in step (b) and corresponding indicator in step (d) can be related molecules, such as two different fluorophores. In one embodiment, the level of the indicator which correlates with modulation of the DP receptor variant in step (b) is greater than the level of the corresponding indicator from step (d). In another embodiment, the level of the indicator which correlates with modulation of the DP receptor variant in step (b) is less than the level of the corresponding indicator from step (d).

The invention also provides a method for identifying a compound that differentially binds to a DP receptor variant by a) contacting an isolated DP receptor variant or a DP receptor variant over-expressed in a genetically engineered cell with a compound; b) determining specific binding of the compound to the DP receptor variant; c) contacting a second receptor with the compound; d) determining specific binding of the compound to the second receptor; and e) comparing the level of specific binding from step (b) with the level of specific binding from step (d), where a different level of specific binding from step (b) compared to the level of specific binding from step (d) indicates that the compound is a compound that differentially binds to the DP receptor variant, and where the DP receptor variant contains an amino acid sequence having at least 50% amino acid identity with SEQ ID NO: 4 and contains the amino acid sequence of SEQ ID NO: 5 or 6, or a conservative variant thereof. In one embodiment, the different level of specific binding is an increased level of binding. In another embodiment, the different level of specific binding is a decreased level of binding.

As set forth above in regard to methods for identifying a compound that modulates or specifically binds a DP receptor variant, the DP receptor variant can be any of a variety of DP receptor variants such as an isolated polypeptide containing an amino acid sequence having at least 50% amino acid identity with SEQ ID NO: 4 and containing the amino acid sequence of SEQ ID NO: 5 or 6, or a conservative variant thereof; or an isolated polypeptide containing the amino acid sequence of SEQ ID NO: 2, or a conservative variant thereof. In addition, the DP receptor variant can be over-expressed in a genetically engineered cell. For example, the DP receptor variant can be exogenously over-expressed in a genetically engineered cell.

In the methods of the invention for identifying a compound that differentially modulates or differentially binds a DP receptor variant, the second receptor can be any receptor of interest. For example, the second receptor can be a G-protein coupled receptor such as, without limitation, any other DP receptor such as a different DP receptor variant or a wild-type DP receptor. In particular embodiments, the second receptor is a wild-type DP receptor containing the amino acid sequence SEQ ID NO: 4, or a functional fragment thereof. The second receptor can be, for example, expressed in a cell endogenously or exogenously or can be an isolated polypeptide.

It is understood that the methods of the invention can be practiced where the DP receptor variant and second receptor are expressed, for example, in different cells. In addition, the methods of the invention can be practiced where the DP receptor variant and second receptor are expressed in the same cell, for example, where the DP receptor variant does not have identical binding and signal transduction effects as the co-expressed second receptor.

The invention also provides an isolated nucleic acid molecule having a nucleotide sequence that encodes a polypeptide containing an amino acid sequence having at least 50% amino acid identity with SEQ ID NO: 4 and containing the amino acid sequence of SEQ ID NO: 5 or 6, or a conservative variant thereof. The invention also provides an isolated nucleic acid molecule having a nucleotide sequence that encodes a polypeptide containing an amino acid sequence having at least 80% or at least 90% amino acid identity with SEQ ID NO: 4 and containing the amino acid sequence of SEQ ID NO: 5 or 6, or a conservative variant thereof. The invention further provides an isolated nucleic acid molecule containing a nucleotide sequence that encodes the amino acid sequence of SEQ ID NO: 2, or a conservative variant thereof. The invention also provides an isolated nucleic acid molecule containing a nucleotide sequence that encodes SEQ ID NO: 2, such as the nucleotide sequence of SEQ ID NO: 1.

Isolated nucleic acid molecules include DNA and RNA molecules as well as both sense or complementary anti-sense strands. It is understood that an isolated nucleic acid molecule of the invention can be a double-stranded or single-stranded molecule, an RNA or DNA molecule, and can optionally include non-coding sequence. DNA molecules of the invention include cDNA sequences as well as wholly or partially chemically synthesized DNA sequences.

The nucleic acid molecules of the invention optionally include heterologous nucleic acid sequences that are not part of the DP receptor variant-encoding sequences in nature. Such a heterologous nucleic acid sequence can be optionally separated from the DP receptor variant-encoding sequence by an encoded cleavage site that facilitates removal of non-DP receptor variant polypeptide sequences from the expressed fusion protein. Heterologous nucleic acid sequences include, without limitation, sequences encoding poly-histidine sequences, FLAG tags and other epitopes, and glutathione-S-transferase, thioredoxin, and maltose binding protein domains or other domains or sequences that facilitate purification or detection of the fusion protein containing a DP receptor variant of the invention.

The location of exons from the human DP receptor genomic clone AL365475 that are present in alternatively spliced human DP receptor variant DP VAR-1 as determined using BLAST searches indicate that the DP receptor variant VAR-1 alternatively spliced sequence corresponds to human genomic clone AL365475 at a range from +102332 to +102242.

The invention further provides a vector containing a nucleic acid molecule having a nucleotide sequence that encodes a polypeptide containing an amino acid sequence having at least 50% amino acid identity with SEQ ID NO: 4 and containing the amino acid sequence of SEQ ID NO: 5 or 6, or a conservative variant thereof. The invention also provides a vector containing a nucleic acid molecule containing a nucleotide sequence that encodes the amino acid sequence of SEQ ID NO: 2, or a conservative variant thereof. In addition, the invention also provides a vector containing a nucleic acid molecule containing or consisting of SEQ ID NO: 2. For example, such a vector can contain a nucleic acid molecule having the nucleotide sequence of SEQ ID NO: 1. The invention further provides a host cell including a vector which contains a nucleic acid molecule of the invention.

Vectors are useful, for example, for subcloning and amplifying a nucleic acid molecule encoding a polypeptide of the invention and for recombinantly expressing the encoded DP variant receptor or other polypeptide. Vectors of the invention include, without limitation, viral vectors such as a bacteriophage, baculovirus and retrovirus vectors; cosmids or plasmids; and, particularly for cloning large nucleic acid molecules, bacterial artificial chromosome vectors (BACs) and yeast artificial chromosome vectors (YACs). Such vectors are commercially available, and their uses are well known in the art. Vectors further encompass expression vectors such as those discussed herein above.

As understood by one skilled in the art, a nucleic acid molecule of the invention can contain nucleotide sequence in addition to nucleotide sequence encoding a DP variant polypeptide of the invention. For example, a nucleic acid molecule of the invention can include one or more additional heterologous sequences such as nucleotide sequences encoding restriction enzyme sites or epitope tags. As non-limiting examples, nucleic acid molecules of the invention can be used in hybridization reactions such as Southern and Northern blots, to encode polypeptide sequence in recombinant cloning methods, or as primers in polymerase chain reactions.

The invention also provides an isolated nucleic acid molecule having a nucleotide sequence that encodes a polypeptide containing or consisting of substantially the same amino acid sequence as SEQ ID NO: 2. For example, the invention provides an isolated nucleic acid molecule having the nucleotide sequence of SEQ ID NO: 1.

The invention further provides a method for preventing or reducing the severity of a disease associated with a DP receptor or a DP receptor variant in a subject by introducing into the subject a compound that modulates or specifically binds to a DP receptor variant or another compound identified by a method of the invention. The invention also provides a method for regulating pain in a subject by introducing into the subject a compound that modulates or specifically binds to a DP receptor variant or another compound identified by a method of the invention. In addition, the invention provides a method for preventing or reducing the severity of a sleep disorder, an ocular condition such as glaucoma or an allergic disorder such as allergic asthma or allergic rhinitis in a subject by introducing into the subject a compound that modulates or specifically binds to a DP receptor variant or another compound identified by a method of the invention.

As used herein, a “disease associated with a DP receptor or DP receptor variant” means any disease or condition in which modulation of the activity of the wild-type DP receptor or a DP receptor variant can be beneficial. It is understood that the underlying cause of the disease may or may not be due to an abnormality in expression or activity of a wild-type DP receptor or DP receptor variant.

Diseases associated with a DP receptor or DP receptor variant encompass, without limitation, pain; ocular conditions including various retinopathies, glaucoma and ocular hypertension; allergic disorders such as, without limitation, allergic asthma and allergic rhinitis; gastrointestinal disorders including, without limitation, diarrhea, irritable bowel syndrome, Crohn's disease and other diseases involving gastrointestinal inflammation; and sleep disorders. Additional diseases or conditions associated with a DP receptor or a DP receptor variant can include, without limitation, diseases involving the spleen, colon or small intestine. As non-limiting examples, a compound that modulates a DP receptor or DP receptor variant can be used in humans for prophylactic or other treatment of pain; an ocular condition such as glaucoma or ocular hypertension; an allergic disorder; or sleep disorder.

A compound that modulates or differentially modulates a DP receptor variant or another compound identified by a method of the invention also can be useful for preventing or treating pain. The term pain, as used herein, includes, without limitation, inflammatory pain, headache pain, muscle pain, visceral pain, neuropathic pain, and referred pain. Pain can be continuous or intermittent, of short duration such as acute pain, or of long duration such as chronic pain. Chronic pain is distinguished from acute pain, which is immediate, generally high threshold, pain brought about by injury such as a cut, crush, burn, or by chemical stimulation such as that experienced upon exposure to capsaicin, the active ingredient in chili peppers.

The methods of the invention further can be used, without limitation, to treat chronic or other headache pain such as pain associated with cluster headaches, tension headaches or chronic daily headaches; muscle pain including, but not limited to, that associated with back or other spasm; inflammatory pain or other symptoms resulting, for example, from spondylitis or arthritis such as rheumatoid arthritis, gouty arthritis, or osteoarthritis; gout; bursitis; painful menstruation and fever. In addition, the methods of the invention can be used, for example, to treat pain associated with injury, surgery, dental procedures, dysmenorrhea, labor and other pain associated with the female reproductive system, and systemic illness such as, without limitation, cancer. It is understood that these and other conditions which may respond to NSAIDs can be prevented or treated using a compound that modulates or differentially modulates a DP receptor variant disclosed herein.

A compound identified by the methods of the invention can be used, without limitation, to prevent or reduce the severity of glaucoma. Glaucoma, the second most common cause of blindness in the United States, affects about two million Americans, but roughly half are unaware of it. This group of disorders is characterized by progressive damage to the eye at least partly due to intraocular pressure. Normal intraocular pressure (IOP) ranges between 11 and 21 mm Hg; however, this level may not necessarily be healthy for all people. Some people with normal pressure develop optic nerve injury (normal- or low-pressure glaucoma). In contrast, many people have pressure greater than 21 mm Hg without any optic nerve injury (ocular hypertension). Of those with ocular hypertension, only about 1% per year will develop glaucoma.

Glaucoma can be described according to the mechanism of outflow obstruction as either open-angle or closed-angle (angle-closure) glaucoma. Alternatively, classification can be based on etiology as primary or secondary. The primary (conventional) outflow system of the eye is located in the anterior chamber angle and accounts for 83 to 96% of aqueous outflow in human eyes under normal circumstances. The primary outflow system refers to aqueous outflow through the trabecular meshwork, canal of Schlemm, intrascleral channels, and episcleral and conjunctival veins. In open-angle glaucoma with elevated intraocular pressure, pressure elevation occurs because outflow is inadequate despite an angle that appears open and relatively normal on gonioscopic examination. In closed-angle glaucoma, elevated intraocular pressure occurs when normal drainage of aqueous fluid from the eye is sufficiently prevented by a physical obstruction of the peripheral iris. The secondary (alternative) aqueous outflow pathways (known as the unconventional or uveoscleral aqueous outflow system) account for 5 to 15% of the total aqueous outflow. The secondary aqueous outflow pathway refers to aqueous exiting the eye through the anterior face of the ciliary body and percolating through the ciliary muscles to the suprachoroidal space (i.e., between the choroid and sclera), where it eventually exits the eye via scleral channels or blood vessels. It is understood that compounds that modulate or specifically bind to a DP receptor variant or that are otherwise identified according to a method of the invention can be used to treat any of a variety of forms of glaucoma including, but not limited to, normal- or low-pressure glaucoma, glaucoma with elevated intraocular pressure, primary glaucoma and secondary glaucoma.

Furthermore, a compound that modulates or specifically binds to a DP receptor variant or which is otherwise identified by a method of the invention can be used alone or in combination with one or more different compounds or other therapeutics or procedures for treatment of glaucoma. Compounds that are currently used in the treatment of glaucoma include, but are not limited to, topical-blockers such as timolol, levobunolol, carteolol, metipranolol and betaxolol; topical nonselective adrenergic agonists such as epinephrine and dipivefrin; adrenergic agonists such as apraclonidine and brimonidine; topical cholinergic agonists such as pilocarpine and phospholine; oral carbonic anhydrase inhibitors such as acetazolamide and methazolamide; topical carbonic anhydrase inhibitors such as dorzolamide; and topical prostaglandin analogs such as latanoprost, unoprostone, and travoprost.

Other ocular conditions that can be prevented or treated with a compound that modulates or differentially modulates a DP receptor variant by a method of the invention include, without limitation, diabetic retinopathy; macular edema such as that associated with diabetes; conditions of retinal degeneration, for example, macular degeneration such as age-related macular degeneration (ARMD) and retinitis pigmentosa; retinal dystrophies; inflammatory disorders of the retina; vascular occlusive conditions of the retina such as retinal vein occlusions or branch or central retinal artery occlusions; retinopathy of prematurity; retinopathy associated with blood disorders such as sickle cell anemia; elevated intraocular pressure; ocular itch; damage following retinal detachment; damage or insult due to vitrectomy, retinal or other surgery; and other retinal damage including therapeutic damage such as that resulting from laser treatment of the retina, for example, pan-retinal photocoagulation for diabetic retinopathy or photodynamic therapy of the retina, for example, for age-related macular degeneration. Ocular conditions that can be prevented or treated with a compound that modulates or differentially modulates a DP receptor variant by a method of the invention further include, without limitation, genetic and acquired optic neuropathies such as optic neuropathies characterized primarily by loss of central vision, for example, Leber's hereditary optic neuropathy (LHON), autosomal dominant optic atrophy (Kjer disease) and other optic neuropathies such as those involving mitochondrial defects, aberrant dynamin-related proteins or inappropriate apoptosis; and optic neuritis such as that associated with multiple sclerosis, retinal vein occlusions or photodynamic or laser therapy. See, for example, Carelli et al., Neurochem. Intl. 4 0:573-584 (2002); and Olichon et al., J. Biol. Chem. 278:7743-7746 (2003).

As described above, a compound that modulates or differentially modulates a DP receptor variant or another compound identified by a method of the invention also can be useful for preventing or treating an allergic disorder or other immune disease. Such a compound can be used to prevent or treat a disorder such as, without limitation, asthma such as allergic asthma, allergic conjunctivitis or allergic rhinitis.

In the methods of the invention for preventing or reducing the severity of pain or glaucoma or another disease associated with a DP receptor or DP receptor variant, a compound can optionally be formulated together with a pharmaceutically acceptable carrier for delivery to the subject to be treated. Suitable pharmaceutically acceptable carriers are well known in the art and include, for example, aqueous or organic solvents such as physiologically buffered saline, glycols, glycerol, oils or injectable organic esters. A pharmaceutically acceptable carrier can also contain a physiologically acceptable agent that acts, for example, to stabilize or increase solubility of a pharmaceutical composition. Such a physiologically acceptable agent can be, for example, a carbohydrate such as glucose, sucrose or dextrans; an antioxidant such as ascorbic acid or glutathione; a chelating agent; a low molecular weight polypeptide; or another stabilizer or excipient. Pharmaceutically acceptable carriers including solvents, stabilizers, solubilizers and preservatives, are well known in the art as described, for example, in Martin, Remington's Pharm. Sci., 15th Ed. (Mack Publ. Co., Easton, 1975).

Ophthalmic compositions can be useful in the methods of the invention for preventing or alleviating an ocular condition. An ophthalmic composition contains an ophthalmically acceptable carrier, which is any carrier that has substantially no long term or permanent detrimental effect on the eye to which it is administered. Examples of ophthalmically acceptable carriers include, without limitation, water, such as distilled or deionized water; saline; and other aqueous media.

Topical ophthalmic compositions useful for alleviating an ocular condition include, without limitation, ocular drops, ocular ointments, ocular gels and ocular creams. Such ophthalmic compositions are easy to apply and deliver the active compound effectively.

A preservative can be included, if desired, in an ophthalmic composition useful in a method of the invention. Such a preservative can be, without limitation, benzalkonium chloride, chlorobutanol, purite, thimerosal, phenylmercuric acetate, or phenylmercuric nitrate. Vehicles useful in a topical ophthalmic composition include, yet are not limited to, polyvinyl alcohol, povidone, hydroxypropyl methyl cellulose, poloxamers, carboxymethyl cellulose, hydroxyethyl cellulose and purified water.

A tonicity adjustor also can be included, if desired, in an ophthalmic composition administered to alleviate an ocular condition without concomitant sedation according to a method of the invention. Such a tonicity adjustor can be, without limitation, a salt such as sodium chloride, potassium chloride, mannitol or glycerin, or another pharmaceutically or ophthalmically acceptable tonicity adjustor.

Various buffers and means for adjusting pH can be used to prepare an ophthalmic composition useful in the invention, provided that the resulting preparation is ophthalmically acceptable. Such buffers include, but are not limited to, acetate buffers, citrate buffers, phosphate buffers and borate buffers. It is understood that acids or bases can be used to adjust the pH of the composition as needed. Ophthalmically acceptable antioxidants useful in preparing an ophthalmic composition include, yet are not limited to, sodium metabisulfite, sodium thiosulfate, acetylcysteine, butylated hydroxyanisole and butylated hydroxytoluene.

Those skilled in the art can formulate a compound that modulates, differentially modulates, specifically binds, or differentially binds a DP receptor variant to ensure proper compound distribution and bioavailablility in vivo. For example, some regions of the eye can be inaccessible to some systemically administered drugs, and as a result topical drug delivery can be used. Polymers can be added to ophthalmic solutions to increase bioavailability (Ludwig and Ootenhgm, S.T.P. Pharm. Sci., 2:81-87 (1992)). In addition, colloidal systems such as, without limitation, liposomes, microparticles or nanoparticules can be used to increase penetration of a compound into the eye. Ocular drug absorption also can be enhanced using, for example, iontophoresis, prodrugs, and cyclodextrins.

Methods of ensuring appropriate distribution in vivo also can be provided by rechargeable or biodegradable devices, particularly where concentration gradients or continuous delivery is desired. Various slow release polymeric devices are known in the art for the controlled delivery of drugs, and include both biodegradable and non-degradable polymers and hydrogels. Polymeric device inserts can allow for accurate dosing, reduced systemic absorption and in some cases, better patient compliance resulting from a reduced frequency of administration. Those skilled in the art understand that the choice of the pharmaceutical formulation and the appropriate preparation of the compound will depend on the intended use and mode of administration.

A compound that modulates or specifically binds to a DP receptor variant, or that is otherwise identified by a screening method of the invention can be administered to a subject by any effective route. Suitable routes of administration include, but are not limited to, oral, sublingual, topical, intraocular, intradermal, parenteral, intranasal, intravenous, intramuscular, intraspinal, intracerebral and subcutaneous routes. The present invention also provides compounds containing an acceptable carrier such as any of the standard pharmaceutical carriers, including phosphate buffered saline solution, water and emulsions such as an oil and water emulsion, and various types of wetting agents.

A method of the invention is practiced by peripherally administering to a subject an effective amount of a compound that modulates or differentially modulates a DP receptor variant or another compound identified by a method of the invention. As used herein in reference to such a compound, the term “peripherally administering” or “peripheral administration” means introducing the compound into a subject outside of the central nervous system. Thus, peripheral administration encompasses any route of administration other than direct administration to the spine or brain.

An effective amount of a compound of the invention can be peripherally administered to a subject by any of a variety of means depending, for example, on the type of condition to be alleviated, the pharmaceutical formulation, and the history, risk factors and symptoms of the subject. Routes of peripheral administration suitable for the methods of the invention include both systemic and local administration. As non-limiting examples, an effective amount of a compound of the invention can be administered orally; sublingually; parenterally; by subcutaneous pump; by dermal patch; by intravenous, intra-articular, subcutaneous or intramuscular injection; by topical drops, creams, gels or ointments; as an implanted or injected extended release formulation; or by subcutaneous minipump or other implanted device, and by inhalation by aerosol and similar devices.

One skilled in the art understands that peripheral administration can be local or systemic. Local administration results in significantly more of a compound of the invention being delivered to and about the site of local administration than to regions distal to the site of administration. Systemic administration results in delivery of a compound of the invention essentially throughout at least the entire peripheral system of the subject.

Routes of peripheral administration useful in the methods of the invention encompass, without limitation, oral administration, sublingual administration, topical administration, intravenous or other injection, and implanted minipumps or other extended release devices or formulations. A compound of the invention can be peripherally administered, without limitation, orally in any acceptable form such as in a tablet, pill, capsule, powder, liquid, suspension, emulsion or the like; an aerosol; as a suppository; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation. A compound of the invention optionally can be packaged in unit dosage form suitable for single administration of precise dosages, or in sustained release dosage form for continuous controlled administration.

Chronic pain and other chronic conditions such as, without limitation, chronic neurological conditions can be prevented or treated using any of a variety of forms of repeated or continuous administration as necessary. In the methods of the invention for alleviating chronic pain or another chronic condition, means for repeated or continuous peripheral administration include, without limitation, repeated oral or topical administration, and administration via subcutaneous minipump. As non-limiting examples, a method of the invention can be practiced by continuous intravenous administration via implanted infusion minipump, or using an extended release formulation.

It is understood that slow-release formulations can be useful in the methods of the invention for alleviating chronic pain or other chronic conditions such as, without limitation, a chronic neurodegenerative conditions. It is further understood that the frequency and duration of dosing will be dependent, in part, on the alleviation desired and the half-life of the compound of the invention and that a variety of routes of administration are useful for delivering slow-release formulations, as detailed herein above.

A compound of the invention can be peripherally administered to a subject to alleviate an ocular condition by any of a variety of means depending, in part, on the characteristics of the compound to be administered and the history, risk factors and symptoms of the subject. Peripheral routes of administration suitable for alleviating an ocular condition in a method of the invention include both systemic and local administration. In particular embodiments, a pharmaceutical composition containing a compound of the invention is administered topically, or by local injection, or is released from an intraocular or periocular implant.

Systemic and local routes of administration useful in alleviating an ocular condition according to a method of the invention encompass, without limitation, oral gavage; intravenous injection; intraperitoneal injection; intramuscular injection; subcutaneous injection; transdermal diffusion and electrophoresis; topical eye drops and ointments; periocular and intraocular injection including subconjunctival injection; extended release delivery devices such as locally implanted extended release devices; and intraocular and periocular implants including bioerodible and reservoir-based implants.

In one embodiment, a method of the invention for alleviating an ocular condition is practiced by administering an ophthalmic composition containing a compound of the invention topically to the eye. The compound can be administered, for example, in an ophthalmic solution (ocular drops). In another embodiment, an ophthalmic composition containing a compound of the invention is injected directly into the eye. In a further embodiment, an ophthalmic composition containing a compound of the invention is released from an intraocular or periocular implant such as a bioerodible or reservoir-based implant.

As indicated above, an ophthalmic composition containing a compound of the invention can be administered locally via an intraocular or periocular implant, which can be, without limitation, bioerodible or reservoir-based. An implant refers to any material that does not significantly migrate from the insertion site following implantation. An implant can be biodegradable, non-biodegradable, or composed of both biodegradable and non-biodegradable materials; a non-biodegradable implant can include, if desired, a refillable reservoir. Implants useful in a method of the invention for alleviating an ocular condition include, for example, patches, particles, sheets, plaques, microcapsules and the like, and can be of any shape and size compatible with the selected site of insertion, which can be, without limitation, the posterior chamber, anterior chamber, suprachoroid or subconjunctiva of the eye. It is understood that an implant useful in the invention generally releases the implanted pharmaceutical composition at an effective dosage to the eye of the subject over an extended period of time. A variety of ocular implants and extended release formulations suitable for ocular release are well known in the art, as described, for example, in U.S. Pat. Nos. 5,869,079 and 5,443,505.

An effective dose of a compound for use in a method of the invention can be determined, for example, by extrapolation from the concentration required in a DP receptor or DP receptor variant binding or activity assay such as one of the assays disclosed herein above. An effective dose of a compound for the treatment of a disease associated with a DP receptor or DP receptor variant also can be determined from appropriate animal models, such as transgenic mice. As non-limiting examples, animal models for pathologies such as cardiovascular disease and ocular diseases are well-known in the art. An effective dose for preventing or reducing the severity of a disease is a dose that results in either partial or complete alleviation of at least one symptom of the disease. The appropriate dose of a compound for treatment of a human subject can be determined by those skilled in the art, and is dependent, for example, on the particular disease being treated, nature and bioactivity of the particular compound, the desired route of administration, the gender, age and health of the individual, and the number of doses and duration of treatment.

All journal article, reference and patent citations provided herein, including referenced sequence accession numbers of nucleotide and amino acid sequences contained in various databases, in parentheses or otherwise, whether previously stated or not, are incorporated herein by reference in their entirety.

It is understood that modifications which do not substantially affect the activity of the various embodiments of this invention are also included within the definition of the invention provided herein. Accordingly, the following examples are intended to illustrate but not limit the present invention.

### EXAMPLE I

### Identification of an Alternatively Spliced DP Receptor Variant

This example describes the molecular cloning of the alternatively spliced DP receptor variant DP VAR-1 and its expression in cell culture.

Human heart, brain, lung, spleen, small intestine, skeletal muscle, kidney and liver total RNA were purchased from Clontech. Total RNA was isolated from human eyes (NDRI; Philadelphia, Pa.) and human ocular tissues (ciliary smooth muscles, trabecular meshwork, ODM-2) using a Qiagen total RNA isolation kit according to the manufacturer's instructions. The ODM-2 cell line is derived from human non-pigmented ciliary epithelial cells (Exchribano et al., J. Cell Physiol. 160:511-521 (1994)). Using 5 μg of human total RNA, first strand cDNA was synthesized by SuperScript II RNase H reverse transcriptase (Life Technologies). Subsequently, 20 μl reactions containing 5 μg of RNA, 250 ng of oligo (dT), and 100 units of reverse transcriptase were incubated at 42° C. for 1 hour and terminated by 100° C. for 3 minutes.

The PCR buffer contained 10 mM Tris-HCl, pH 8.3, 50 mM KCl, 2 mM MgCl₂, 2.5 units AmpliTaq DNA polymerase and 0.2 μM upstream and downstream primers in a final volume of 50 μl. After an initial incubation for 5 min at 94° C., samples were subjected to 30 cycles of 30 seconds at 95° C., 30 seconds at 58° C., and 30 seconds at 72° C. in a PE 9700 thermal cycler. The primers used for the detection of human DP alternative splice variants were as follows: Human DP forward (TGATGACCGTGCTCTTCACT; SEQ ID NO: 11) and human DP reverse (GATAGAAATCGCAAGGCTCG; SEQ ID NO: 12).

For subsequent analysis, PCR products were isolated from 1.5% lower melting agarose gel and subcloned into TOPO PCRII vector (Invitrogen; Carlsbad, Calif.). Sequence analysis was performed by Sequetech (Mountain View, Calif.).

Full length cDNA for the DP receptor variant DP VAR-1 was isolated and subcloned into TOPO pcDNA3.1 PCR cloning vector (Invitrogen; Carlsbad, Calif.) or pCEP4 expression vector (Invitrogen) to create DP VAR-1/pcDNA3.1 plasmids or DP VAR-1/pCEP4 plasmids. DP VAR-1/pcDNA3.1 plasmids were used for transient transfection, and DP VAR-1/pCEP4 plasmids were used for stable transfection. Full length Gα₁₆ cDNA was subcloned into the pcDNA3.1 vector. The plasmids were sequenced by Sequetech.

HEK 293/EBNA cells were obtained from the American Type Culture Collection (ATCC). HEK 293/EBNA cells were routinely maintained in DMEM with 10% fetal bovine serum, 1% glutamine, 0.5% penicillin/streptomycin. Cells were kept in humidified 5% CO₂, 95% air at 37° C. For stable transfection, DP VAR-1/pCEP4 plasmids were transfected into HEK 293/EBNA cells using Fugene 6 (Roche Diagnostics Corp., Inc.; Indianapolis, Ind.), according to the manufacture's instructions. Selection for cell clones that stably expressed the plasmid was performed with 200 μg/ml hygromycin.

### EXAMPLE II

### Tissue Distribution of Alternatively Spliced DP Receptor Variant DP VAR-1

This example shows the tissue distribution of the alternatively spliced DP receptor variant DP VAR-1.

Total RNA was isolated from human ciliary body using RNeasy Kit (Qiagen, Inc.; Valencia, Calif.) according to the manufacturer's instructions. RNA concentrations were determined by UV Spectrophotometry (Beckman DU640) at A 260 nM, and stored at −80° C.

Other human total RNAs were purchased from Clontech. Multiple tissue RT-PCR analysis was performed to detect the alternatively spliced DP mRNA using primers SEQ ID NOS: 11 and 12 which are specific to the alternatively spliced DP VAR-1 and the PCR conditions described in Example I above.

Using human alt DP receptor specific primers, RT-PCR analysis was performed. As shown in FIG. 3, the results indicate that the DP VAR-1 mRNA is specifically expressed in small intestine and spleen.

These results indicate that the DP VAR-1 is an alternatively spliced transcript which is expressed in a tissue-specific fashion.

### EXAMPLE III

### Screening Assays using DP Receptor Variants

This example describes a FLIPR and luciferase assay for screening compounds against DP receptor variants.

HEK 293/EBNA cells transiently or stably expressing DP VAR-1 /pcDNA3.1 plasmids were seeded at a density of 5×10³ cells per well in Biocoat Poly-D-lysine-coated black-wall, clear-bottom 96-well plates (Becton-Dickinson; Franklin Lakes, N.J.) and allowed to attach overnight. At 48 hours after transfection, the cells were washed two times with HBSS-HEPES buffer (Hanks Balanced Salt Solution without bicarbonate and phenol red, 20 mM HEPES, pH 7.4) using a Lab Systems Cellwash plate washer. After 45 minutes of dye-loading in the dark, using the calcium-sensitive dye Fluo-4 AM at a final concentration of 2 mM, the plates were washed four times with HBSS-HEPES buffer to remove excess dye leaving 100 μl in each well. Plates were re-equilibrated to 37° C. for a few minutes. The cells were excited with an Argon laser at 488 nm, and emission was measured through a 510-570 nm bandwidth emission filter (FLIPR; Molecular Devices; Sunnyvale, Calif.). Compound solution was added in a 50 μl volume to each well to give the desired final concentration. The peak increase in fluorescence intensity was recorded for each well. To generate concentration-response curves, compounds were tested in duplicate in a concentration range between 10⁻¹¹ and 10⁻⁵ M. Duplicate values were averaged.

CRE-luciferase reporter plasmids purchased from Invitrogen were used for detecting cAMP accumulation in Gas coupled receptors. pGL3-N-960 plasmids containing human Nur77 promoter (Uemura et al., J. Biol. Chem. 270:5427-5433 (1995)) and pGL3-CTGF-LUC plasmids containing human CTGF promoter were used for detecting calcium, PKC, and MAP kinase pathways associated with G_{αq} coupled receptors. For the pGL3-CTGF-LUC plasmid, a DNA fragment containing the CTGF promoter region from −2047 to +65 (Fu et al., J. Biol. Chem 276:45888-45894 (2001)) was cloned from human genomic DNA (Clontech). The fragment was subcloned into a pGL3 luciferase expression vector (Promega Inc.) creating the pGL3-CTGF-LUC plasmid.

Luciferase reporter plasmids were transfected into HEK 293/EBNA cells transiently or stably expressing DP receptor variants using Fugene 6, according to the manufacturer's instructions. In brief, the cells were plated in 24 well plates overnight, and then the 24 well plate cells were washed twice and resuspended in 1 ml of DMEM. The cell suspension was mixed with 0.2 μg of plasmid DNA in 100 μl of DMEM containing 0.6 μl Fugene 6 solution and added into each well. Plates were cultured for 24 hours at 37° C. before compounds were added to the cultures at concentrations ranging from 10⁻¹¹ to 10⁻⁶ M. Cells were harvested 6 hours later and lysed in 100 μl of 25 mM Tris-phosphate buffer (pH 7.5) containing 1% Triton X-100. Soluble extracts (20 μl) were assayed for luciferase activity as described below.

The luciferase assay was performed with a Promega assay kit (Promega, Inc.; Madison, Wis.) at room temperature using an Autolumat LB 953 (Berthold; Bad Wildbad, Germany). Luciferase content was measured by calculating the light emitted during the initial 10 seconds of the reaction. Relative luciferase activity was expressed as fold values of ratio compared to control. Experiments were independently repeated at least 3 times.

All journal article, reference and patent citations provided herein, including referenced sequence accession numbers of nucleotide and amino acid sequences contained in various databases, in parentheses or otherwise, whether previously stated or not, are incorporated herein by reference in their entirety.

Although the invention has been described with reference to the disclosed embodiments, those skilled in the art will readily appreciate that the specific experiments detailed are only illustrative of the invention. It should be understood that various modifications can be made without departing from the spirit of the invention.

1. An isolated polypeptide, comprising
(a) an amino acid sequence having at least 50% amino acid identity with SEQ ID NO: 4, and
(b) an amino acid sequence of SEQ ID NO: 5 or 6; or a conservative variant thereof.

2. The isolated polypeptide of claim 1, wherein said polypeptide comprises an amino acid sequence having at least 80% amino acid identity with SEQ ID NO: 4.

3. The isolated polypeptide of claim 1, wherein said polypeptide comprises an amino acid sequence having at least 90% amino acid identity with SEQ ID NO: 4.

4. An isolated polypeptide, comprising SEQ ID NO: 2, or a conservative variant thereof.

5. The isolated polypeptide of claim 4, wherein said polypeptide comprises SEQ ID NO: 2.

6. The isolated polypeptide of claim 5, wherein said polypeptide consists of SEQ ID NO: 2.

7. A cell, comprising the exogenously expressed polypeptide of claim 1, 2 or 4.

8. A method for identifying a compound that modulates a DP receptor variant, comprising:
(a) contacting said DP receptor variant with a compound, wherein said DP receptor variant is an isolated DP receptor variant or a DP receptor variant over-expressed in a genetically engineered cell, and
(b) determining the level of an indicator which correlates with modulation of the DP receptor variant,
(c) wherein an alteration in the level of said indicator as compared to a control level indicates that said compound is a compound that modulates the DP receptor variant,
wherein said DP receptor variant is the polypeptide of claim 1.

9. The method of claim 8, wherein said alteration is an increase in the level of said indicator.

10. The method of claim 8, wherein said alteration is a decrease in the level of said indicator.

11. The method of claim 8, wherein said DP receptor variant is a polypeptide comprising an amino acid sequence having at least 80% amino acid identity with SEQ ID NO: 4.

12. The method of claim 8, wherein said DP receptor variant is a polypeptide comprising the amino acid sequence of SEQ ID NO: 2, or a conservative variant thereof.

13. The method of claim 8, wherein said DP receptor variant is an isolated polypeptide.

14. The method of claim 8, wherein said DP receptor variant is a DP receptor variant over-expressed in a genetically engineered cell.

15. The method of claim 14, wherein said DP receptor variant is exogenously expressed.

16. The method of claim 8, wherein said indicator is calcium.

17. The method of claim 8, wherein said compound is a polypeptide.

18. The method of claim 8, wherein said compound is a small molecule.

19. A method for identifying a compound that specifically binds to a DP receptor variant, comprising:
(a) contacting said DP receptor variant with a compound, wherein said DP receptor variant is an isolated DP receptor variant or a DP receptor variant over-expressed in a genetically engineered cell, and
(b) determining specific binding of said compound to said DP receptor variant,
wherein said DP receptor variant is the polypeptide of claim 1.

20. The method of claim 19, wherein said DP receptor variant is a polypeptide comprising an amino acid sequence having at least 80% amino acid identity with SEQ ID NO: 4.

21. The method of claim 19, wherein said DP receptor variant is a polypeptide having the amino acid sequence of SEQ ID NO: 2 or a conservative variant thereof.

22. The method of claim 19, wherein said DP receptor variant is an isolated polypeptide.

23. The method of claim 19, wherein said DP receptor is a DP receptor variant over-expressed in a genetically engineered cell.

24. The method of claim 23, wherein said DP receptor variant is exogenously expressed.

25. The method of claim 19, wherein said contacting occurs in vitro.

26. The method of claim 19, wherein said compound is a polypeptide.

27. The method of claim 19, wherein said compound is a small molecule.

28. A method for identifying a compound that differentially modulates a DP receptor variant, comprising:
(a) contacting said DP receptor variant with a compound, wherein said DP receptor variant is an isolated DP receptor variant or a DP receptor variant over-expressed in a genetically engineered cell;
(b) determining the level of an indicator which correlates with modulation of said DP receptor variant;
(c) contacting a second receptor with said compound;
(d) determining the level of a corresponding indicator which correlates with modulation of said second receptor; and
(e) comparing the level of the indicator from step (b) with the level of the corresponding indicator from step (d), wherein a different level of the indicator from step (b) compared to the level of the corresponding indicator from step (d) indicates that said compound is a compound that differentially modulates said DP receptor variant,
wherein said DP receptor variant is the polypeptide of claim 1.

29. The method of claim 28, wherein said second receptor comprises the amino acid sequence of SEQ ID NO: 4 or a functional fragment thereof.

30. The method of claim 29, wherein said second receptor comprises the amino acid sequence of SEQ ID NO: 4.

31. The method of claim 28, wherein the level of said indicator from step (b) is greater than the level of said corresponding indicator from step (d).

32. The method of claim 28, wherein the level of said indicator from step (b) is less than the level of said corresponding indicator from step (d).

33. The method of claim 28, wherein said DP receptor variant is a polypeptide comprising an amino acid sequence having at least 80% amino acid identity with SEQ ID NO: 4.

34. The method of claim 28, wherein said DP receptor variant is a polypeptide comprising the amino acid sequence SEQ ID NO: 2 or a conservative variant thereof.

35. The method of claim 28, wherein said DP receptor variant is an isolated polypeptide.

36. The method of claim 28, wherein said DP receptor variant is a DP receptor variant over-expressed in a genetically engineered cell.

37. The method of claim 36, wherein said DP receptor variant is exogenously expressed.

38. The method of claim 28, wherein said indicator in step (b) is calcium.

39. The method of claim 28, wherein said compound is a polypeptide.

40. The method of claim 28, wherein said compound is a small molecule.

41. A method for identifying a compound that differentially binds to a DP receptor variant, comprising:
(a) contacting said DP receptor variant with a compound, wherein said DP receptor variant is an isolated DP receptor variant or a DP receptor variant over-expressed in a genetically engineered cell;
(b) determining specific binding of said compound to said DP receptor variant;
(c) contacting a second receptor with said compound;
(d) determining specific binding of said compound to said second receptor; and
(e) comparing the level of specific binding from step (b) with the level of specific binding from step (d), wherein a different level of specific binding from step (b) compared to the level of specific binding from step (d) indicates that said compound is a compound that differentially binds to a DP receptor variant,
wherein said DP receptor variant is the polypeptide of claim 1.

42. The method of claim 41, wherein said second receptor comprises the amino acid sequence of SEQ ID NO: 4, or a functional fragment thereof.

43. The method of claim 41, wherein said second receptor comprises the amino acid sequence of SEQ ID NO: 4.

44. The method of claim 41, wherein said different level of specific binding is an increased level of binding.

45. The method of claim 41, wherein said different level of specific binding is a decreased level of binding.

46. The method of claim 41, wherein said DP receptor variant receptor is a polypeptide comprising an amino acid sequence having at least 80% amino acid identity with SEQ ID NO: 4

47. The method of claim 41, wherein said DP receptor variant is a polypeptide comprising the amino acid sequence of SEQ ID NO: 2 or a conservative variant thereof.

48. The method of claim 41, wherein said DP receptor variant is an isolated polypeptide.

49. The method of claim 41, wherein said DP receptor variant is a DP receptor variant over-expressed in a genetically engineered cell.

50. The method of claim 49, wherein said DP receptor variant is exogenously expressed.

51. The method of claim 41, wherein said contacting occurs in vitro.

52. The method of claim 41, wherein said compound is a polypeptide.

53. The method of claim 41, wherein said compound is a small molecule.


SEQUENCE LISTING (12 sequences)

SEQ ID NO: 1 - DNA, 1171 bp, Homo sapiens
  CDS (1)...(888)
atgaagtcgccgttctaccgctgccagaacaccacctctgtggaaaaaggcaactcggcggtgatgggcggggtgctcttcagcaccggcctcctgggcaacctgctggccctggggctgctggcgcgctcggggctggggtggtgctcgcggcgtccactgcgcccgctgccctcggtcttctacatgctggtgtgtggcctgacggtcaccgacttgctgggcaagtgcctcctaagcccggtggtgctggctgcctacgctcagaaccggagtctgcgggtgcttgcgcccgcattggacaactcgttgtgccaagccttcgccttcttcatgtccttctttgggctctcctcgacactgcaactcctggccatggcactggagtgctggctctccctagggcaccctttcttctaccgacggcacatcaccctgcgcctgggcgcactggtggccccggtggtgagcgccttctccctggctttctgcgcgctacctttcatgggcttcgggaagttcgtgcagtactgccccggcacctggtgctttatccagatggtccacgaggagggctcgctgtcggtgctggggtactctgtgctctactccagcctcatggcgctgctggtcctcgccaccgtgctgtgcaacctcggcgccatgcgcaacctctatgcgatgcaccggcggctgcagcggcacccgcgctcctgcaccagggactgtgccgagccgcgcgcggacgggagggaagcgtcccctcagcccctggaggagctggatcacctcctgctgctggcgctgatgaccgtgctcttcactatgtgttctctgcccgtaattgcatttgttcctggagtccccgccaagacacctgggagtaggtgaggcttgaggaaacattttcagtgctgctcctctcctttctcccaagtatcgcgcttactatggagcatttaaggatgtcaaggagaaaaacaggacctctgaagaagcagaagacctccgagccttgcgatttctatctgtgatttcaattgtggacccttggatttttatcattttcagatctccagtatttcggatattttttcacaagattttcattagacctcttaggtacaggagccggtgcagcaattccactaacatggaatccagtctgtga

SEQ ID NO: 2 - protein, 296 aa, Homo sapiens
MKSPFYRCQNTTSVEKGNSAVMGGVLFSTGLLGNLLALGLLARSGLGWCSRRPLRPLPSVFYMLVCGLTVTDLLGKCLLSPVVLAAYAQNRSLRVLAPALDNSLCQAFAFFMSFFGLSSTLQLLAMALECWLSLGHPFFYRRHITLRLGALVAPVVSAFSLAFCALPFMGFGKFVQYCPGTWCFIQMVHEEGSLSVLGYSVLYSSLMALLVLATVLCNLGAMRNLYAMHRRLQRHPRSCTRDCAEPRADGREASPQPLEELDHLLLLALMTVLFTMCSLPVIAFVPGVPAKTPGSR

SEQ ID NO: 3 - DNA, 1080 bp, Homo sapiens
  CDS (1)...(1080)
atgaagtcgccgttctaccgctgccagaacaccacctctgtggaaaaaggcaactcggcggtgatgggcggggtgctcttcagcaccggcctcctgggcaacctgctggccctggggctgctggcgcgctcggggctggggtggtgctcgcggcgtccactgcgcccgctgccctcggtcttctacatgctggtgtgtggcctgacggtcaccgacttgctgggcaagtgcctcctaagcccggtggtgctggctgcctacgctcagaaccggagtctgcgggtgcttgcgcccgcattggacaactcgttgtgccaagccttcgccttcttcatgtccttctttgggctctcctcgacactgcaactcctggccatggcactggagtgctggctctccctagggcaccctttcttctaccgacggcacatcaccctgcgcctgggcgcactggtggccccggtggtgagcgccttctccctggctttctgcgcgctacctttcatgggcttcgggaagttcgtgcagtactgccccggcacctggtgctttatccagatggtccacgaggagggctcgctgtcggtgctggggtactctgtgctctactccagcctcatggcgctgctggtcctcgccaccgtgctgtgcaacctcggcgccatgcgcaacctctatgcgatgcaccggcggctgcagcggcacccgcgctcctgcaccagggactgtgccgagccgcgcgcggacgggagggaagcgtcccctcagcccctggaggagctggatcacctcctgctgctggcgctgatgaccgtgctcttcactatgtgttctctgcccgtaatttatcgcgcttactatggagcatttaaggatgtcaaggagaaaaacaggacctctgaagaagcagaagacctccgagccttgcgatttctatctgtgatttcaattgtggacccttggatttttatcattttcagatctccagtatttcggatattttttcacaagattttcattagacctcttaggtacaggagccggtgcagcaattccactaacatggaatccagtctgtga

SEQ ID NO: 4 - protein, 359 aa, Homo sapiens
MKSPFYRCQNTTSVEKGNSAVMGGVLFSTGLLGNLLALGLLARSGLGWCSRRPLRPLPSVFYMLVCGLTVTDLLGKCLLSPVVLAAYAQNRSLRVLAPALDNSLCQAFAFFMSFFGLSSTLQLLAMALECWLSLGHPFFYRRHITLRLGALVAPVVSAFSLAFCALPFMGFGKFVQYCPGTWCFIQMVHEEGSLSVLGYSVLYSSLMALLVLATVLCNLGAMRNLYAMHRRLQRHPRSCTRDCAEPRADGREASPQPLEELDHLLLLALMTVLFTMCSLPVIYRAYYGAFKDVKEKNRTSEEAEDLRALRFLSVISIVDPWIFIIFRSPVFRIFFHKIFIRPLRYRSRCSNSTNMESSL

SEQ ID NO: 5 - protein, 10 aa, Homo sapiens
LPVIAFVPGV

SEQ ID NO: 6 - protein, 14 aa, Homo sapiens
AFVPGVPAKTPGSR

SEQ ID NO: 7 - DNA, 12 bp, Homo sapiens
cgtaattgcatt

SEQ ID NO: 8 - DNA, 16 bp, Homo sapiens
tcccgtaattgcattg

SEQ ID NO: 9 - DNA, 12 bp, Homo sapiens
ccaagtatcgcg

SEQ ID NO: 10 - DNA, 16 bp, Homo sapiens
tcccaagtatcgcgcg

SEQ ID NO: 11 - DNA, 20 bp, Artificial Sequence
  primer
tgatgaccgtgctcttcact

SEQ ID NO: 12 - DNA, 20 bp, Homo sapiens
gatagaaatcgcaaggctcg